(19) United States
(12) Patent Application Publication
(10) Pub. No.: US 2026/0207555 A1
(43) Pub. Date: Jul. 23, 2026

(54) TOPICAL KETOROLAC COMPOSITIONS

(71) Applicant: Novilla Pharmaceuticals, Inc., Arlington, VA (US)

(72) Inventor: Eric T. Fossel, Cambridge, MA (US)

(21) Appl. No.: 19/394,749

(22) Filed: Nov. 19, 2025

Related U.S. Application Data
(60) Provisional application No. 63/722,907, filed on Nov. 20, 2024.
(60) Provisional application No. 63/818,266, filed on Jun. 5, 2025.
(60) Provisional application No. 63/867,898, filed on Aug. 21, 2025.

(51) Int. Cl. A61K 31/407; A61K 9/00; A61K 9/107; A61K 47/02; A61K 47/10; A61K 47/14; A61K 47/18; A61K 47/20; A61K 47/22; A61K 47/34; A61K 47/36; A61P 29/00

(52) U.S. Cl. CPC A61K 31/407; A61K 9/0014; A61K 9/107; A61K 47/02; A61K 47/10; A61K 47/14; A61K 47/186; A61K 47/20; A61K 47/22; A61K 47/34; A61K 47/36; A61P 29/00

(57) ABSTRACT

The present disclosure describes topical ketorolac compositions that comprise a eutectic solvent, such as choline chloride and propylene glycol, and methods and uses thereof. The compositions of the present disclosure are capable of delivering an effective amount of ketorolac through the skin and are believed to be useful in treating inflammation and/or pain, such as gout.

### CROSS REFERENCE TO RELATED APPLICATIONS

This application claims priority to U.S. Provisional Application No. 63/722,907, filed Nov. 20, 2024; U.S. Provisional Application No. 63/818,266, filed Jun. 5, 2025; and U.S. Provisional Application No. 63/867,898, filed Aug. 21, 2025, each of which is incorporated herein in its entirety for all purposes.

### BACKGROUND

Ketorolac, sold under the brand name Toradol among others, is a nonsteroidal anti-inflammatory drug (NSAID) approved for treating pain, specifically moderate to severe pain. The mechanism of action is believed to be inhibition of cyclooxygenase 1 (COX1) and cyclooxygenase 2 (COX2), thereby decreasing production of prostaglandins. The approved duration of treatment is less than six days in the United States, and in Switzerland not more than two days. It can be administered by mouth, by nose, by injection into a vein or muscle, or as eye drops. Effects begin within an hour and last for up to eight hours.

Common side effects include sleepiness, dizziness, abdominal pain, swelling, and nausea. Serious side effects may include stomach bleeding, kidney failure, heart attacks, bronchospasm, heart failure, and anaphylaxis. Use is not recommended during the last part of pregnancy or during breastfeeding.

Deep eutectic solvents, also known as eutectic solvents or DES, have a lower melting point than each of their individual components due to ion-dipole interactions and/or hydrogen bonding. DES are believed to be more environmentally friendly because often the components of the mixtures have been toxicologically evaluated. See, e.g., US patent publication numbers US 20210299044 and 20210308048.

Topical administration of drugs can have advantages, such as enhanced delivery of the active ingredient to the local desired site of action and reduced adverse side effects, compared to oral administration. See, e.g., Mehta, et al. Current Opinion Rheumatology 2021, 33 (1): 94-109. For instance, drug delivery systems involving hydrogels, liposomes, and particle-based carriers have been investigated for providing effective relief of osteoarthritis symptoms. Such systems may suffer from high cost of goods in their development.

Drug penetration at therapeutically effective doses, or lack thereof, is a challenge for those in the pharmaceutical industry developing topically administered active ingredients. Many solubilizing agents have come under study, including DESs. Recently, research groups have investigated a variety of DES combinations and poorly-soluble drugs. This class of DESs has been investigated with the goal of overcoming certain shortcomings, such as poor solubility, low absorption rate, and high toxicity, of drugs alone.

The physicochemical properties of a specific active ingredient such as ketorolac must be matched with those of the DES in order to deliver therapeutically effective doses for topical treatment of pain and/or inflammation, such as joint pain. This invention solves this problem among others.

### BRIEF SUMMARY

In some embodiments, the present invention provides a composition comprising:
(a) from about 1% to about 50% (w/w) ketorolac or pharmaceutically acceptable salt thereof;
(b) from about 5% to about 50% (w/w) choline chloride;
(c) from about 20% to about 70% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, the method of the present disclosure is a method of treating inflammation or pain in a human subject in need thereof, comprising topically administering to the subject a composition as described herein.

In some embodiments, the method of the present disclosure is a method of treating inflammation or pain associated with acute gout in a human subject in need thereof, comprising topically administering three times daily to a joint in the subject a therapeutically effective amount of the composition as described herein.

### BRIEF DESCRIPTION OF THE DRAWINGS

FIG. 1 shows calculation of the ketorolac free energy profile within the Long Periodicity Phase (LPP) motif of the Stratum Corneum (SC) modelled for human skin. Y-axis=calculated free energy in kcal/mol; x-axis=LPP depth in Ångstroms.

FIG. 2 shows calculation of active pharmaceutical ingredient (API) distribution within the Long Periodicity Phase (LPP) motif of the Stratum Corneum (SC) modelled for human skin. Y-axis=relative probability; x-axis=LPP depth in Ångstroms.

FIG. 3 shows calculation of entropy of ketorolac and ibuprofen within the Long Periodicity Phase (LPP) motif of the Stratum Corneum (SC) modelled for human skin. Y-axis=entropy in cal/K; x-axis=LPP depth in Angstroms.

FIG. 4 shows calculation of resistance of ketorolac and ibuprofen within the Long Periodicity Phase (LPP) motif of the Stratum Corneum (SC) modelled for human skin. Y-axis=entropy in cal/K; x-axis=LPP depth in Ångstroms.

FIG. 5A-5C show in vitro results for amount of ketorolac permeated through simulated skin over time. FIG. 5A: graph for average cumulative ketorolac transfer for compositions 125001, 125002, 125003, and 125004. Y-axis=ketorolac (μg); x-axis=time (min). FIG. 5B: graph for average cumulative ketorolac transfer (mg, y-axis) vs. time (hours, x-axis) for compositions 125010, 125011, 125012, and 125013. FIG. 5C: graph for average cumulative ketorolac transfer (mg, y-axis) vs. time (hours, x-axis) for compositions 125010, 125016, and 125018.

FIG. 6 shows in vitro results for amount of ketorolac permeated through simulated skin over time for compositions 125010 (“NOV-1776”), and comparative compositions containing an ionic liquid (“IL”) and another eutectic comprising betaine and lactic acid (“BETLA”). Y-axis=ketorolac (mg); x-axis=time (hours).

FIG. 7 shows in vivo results for amount of ketorolac delivered to rat plasma over time for compositions 25002, 25011, 25012, and 25013. Y-axis=ketorolac (μg); x-axis=time (min).

FIG. 8 shows in vivo results in rat pain model for compositions “B” and “C” with control composition “A”. Y-axis=pain withdrawal threshold (PWT) (g); x-axis=time (min).

FIG. 9 shows in vivo results in rat pain model for compositions “B” and “C” with control composition “A”. Y-axis=pain withdrawal threshold (PWT) (g); x-axis=time (min).

FIG. 10 shows in vivo results in single dose pig pharmacokinetics in plasma and synovial fluid for composition 25002. Y-axis=ketorolac concentration (ng/ml); x-axis=time from dosing (min).

FIG. 11 shows in vivo results in single dose pig pharmacokinetics in synovial fluid in inflamed and non-inflamed joint for composition 25002. Y-axis=ketorolac concentration (ng/mL); x-axis=time from dosing (min).

FIG. 12 shows in vivo results in single dose pig pharmacokinetics in synovial fluid of inflamed joint for composition 25002. Y-axis=ketorolac concentration (ng/ml); x-axis=time from dosing (min).

FIG. 13 shows in vivo results in single dose pig pharmacokinetics in synovial fluid for composition 125010. Y-axis=ketorolac concentration (ng/ml); x-axis=time from dosing (min).

FIG. 14 shows in vivo results in multiple dose pig pharmacokinetics in synovial fluid for composition 125010. Y-axis=ketorolac concentration (ng/ml); x-axis=time from dosing (min).

FIG. 15 shows comparison of plasma exposure for composition 125010 as compared to ketorolac IV administration. Y-axis=ketorolac concentration (μg/mL); x-axis=time from dosing (min).

FIG. 16 shows preliminary clinical data for all patients (n=10) after a single topical administration of 5.0 g/dose composition 125010: y-axis=average pain or disability score (from 0-100); x-axis=time (hours).

FIG. 17 shows preliminary clinical data for all patients (n=10) after multiple topical administrations of 5.0 g/dose composition 125010 three times daily over 5 days: y-axis=average pain or disability score (from 0-100); x-axis=time (days).

FIG. 18 shows preliminary clinical data for all patients (n=10) after multiple topical administrations of 5.0 g/dose composition 125010 (“NOV-1776”) three times daily over 5 days: y-axis=percent responders (from 0-100); x-axis=time (hours).

FIG. 19 shows preliminary clinical data for all patients (n=10) after multiple topical administrations of 5.0 g/dose composition 125010 (“NOV-1776”) three times daily over 5 days: average treatment satisfaction score for prior treatment (Day 1) in comparison with treatment satisfaction with composition 125010 (Day 5.5).

FIG. 20 shows preliminary clinical data for all patients (n=10) after multiple topical administrations of 5.0 g/dose composition 125010 (“NOV-1776”) three times daily over 5 days: y-axis=pain score (from 0-100); x-axis=time (hours). Comparison lines show literature pain scores from Roddy E, et al. Ann Rheum Dis 2020; 79:276-284 for colchicine or naproxen treatments.

FIG. 21 shows preliminary clinical data for all patients (n=10) after multiple topical administrations of 5.0 g/dose composition 125010 three times daily over 5 days: y-axis=average number of gout flare duration days; left side points indicate self reports pre-treatment; right side points indicate self reports post-treatment with composition 125010.

FIG. 22A-22B show average plasma ketorolac concentration (ng/ml) in clinical participants: FIG. 22A shows ketorolac plasma concentration dosed once with 30 mg ketorolac intravenous bolus dose as compared to 5.0 g/dose composition 125010 (“NOV-1776”) topically administered three times six hours apart; FIG. 22B shows ketorolac plasma concentration (y-axis, ng/ml) vs. time (hours) in multiple days dosing at 2.1 g/dose or 5.0 g/dose composition 125010 topically administered three times six hours apart.

### DETAILED DESCRIPTION

### I. General

The present disclosure describes topical compositions of ketorolac that comprise a eutectic solvent, such as choline chloride and propylene glycol. Such topical compositions are capable of delivering an effective amount of ketorolac to below the skin surface, e.g., within the synovial fluid of inflamed joints, in order to treat inflammation and/or pain. The topical compositions of the present disclosure are believed advantageous compared to other modes of delivering ketorolac known in the art, such as intravenous delivery of ketorolac, by enhancing local delivery, thereby reducing systemic exposure of ketorolac and reducing potentially toxic effects of high plasma concentration of ketorolac.

Eutectic solvents have been investigated for use in transcutaneous delivery of polar drugs, such as non-steroidal anti-inflammatory drugs (NSAIDs). See, e.g., US patent publication numbers US20210299044 and 20210308048. However, not all eutectic solvent combinations can deliver a therapeutically effective amount of every drug. In fact, the physicochemical properties of the eutectic solvent must be matched with those of the active drug for effective transcutaneous delivery of a sufficient amount of the drug for therapy.

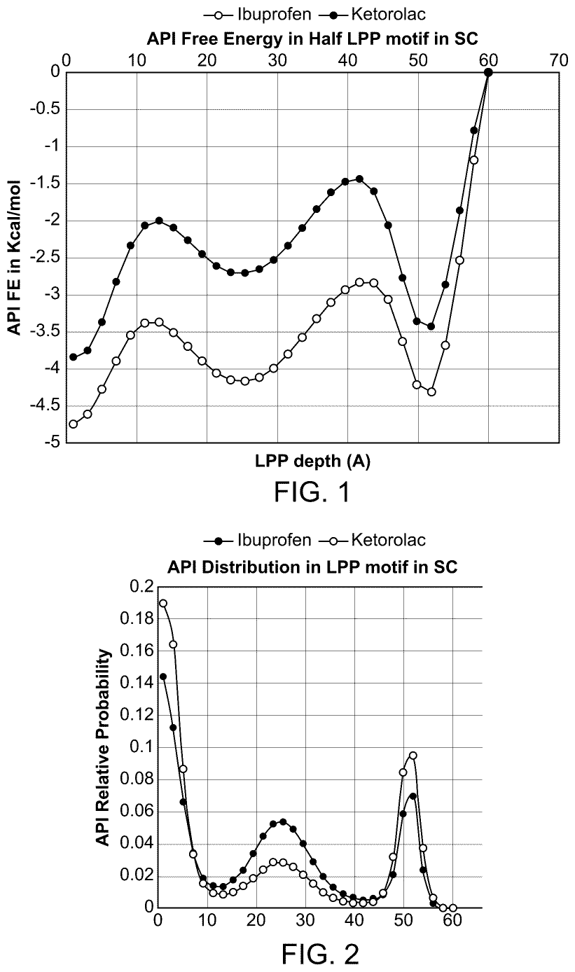

Ketorolac was expected to be difficult to prepare as a therapeutically effective composition containing a eutectic solvent. As illustrated by the in silico comparison with another non-steroidal anti-inflammatory drug (NSAID) ibuprofen, the preparation of a topical ketorolac composition with properties sufficient to deliver an effective therapeutic dose was expected to be more difficult due to its physicochemical properties. See, Example 1 and FIGS. 1-4.

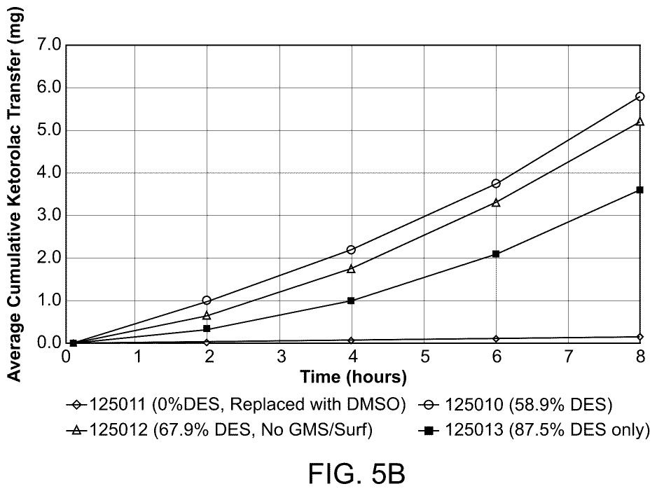

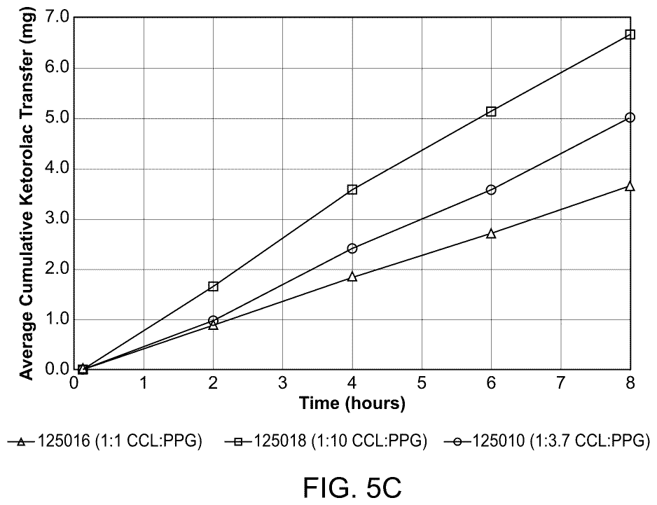

The present invention describes ketorolac compositions containing choline chloride and propylene glycol. See, Example 2. Such compositions are capable of delivering therapeutically effective doses of the active ingredient transcutaneously in in vitro and in vivo studies. See, Examples 3 through 6. Ketorolac compositions of the present disclosure demonstrated effective skin permeation in an in vitro skin model. As illustrated by the compositions of the present disclosure, e.g., Compositions 125010, 125012, and 125013, that each contained ketorolac and a eutectic mixture comprising choline chloride and propylene glycol, performed better for delivering ketorolac across an in vitro membrane than Composition 125011 that contained ketorolac and DMSO only. Further, varying amounts of choline chloride and propylene glycol could be used in a ketorolac composition for effective membrane penetration. See, FIG. 5A-5C. The 125010 ketorolac, choline chloride, and propylene glycol composition as presently disclosed performed better in an in vitro skin permeability model than a comparative ionic liquid or an alternative eutectic composition containing ketorolac, betaine, and lactic acid (FIG. 6).

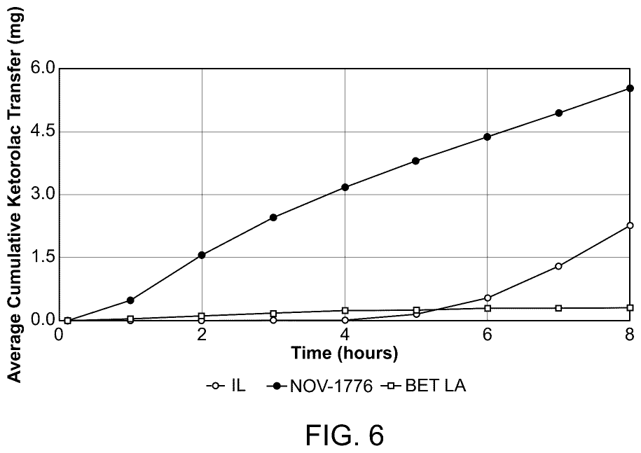

Illustrative ketorolac compositions demonstrated effective treatment of pain in an in vivo rat model. See, FIGS. 7-9.

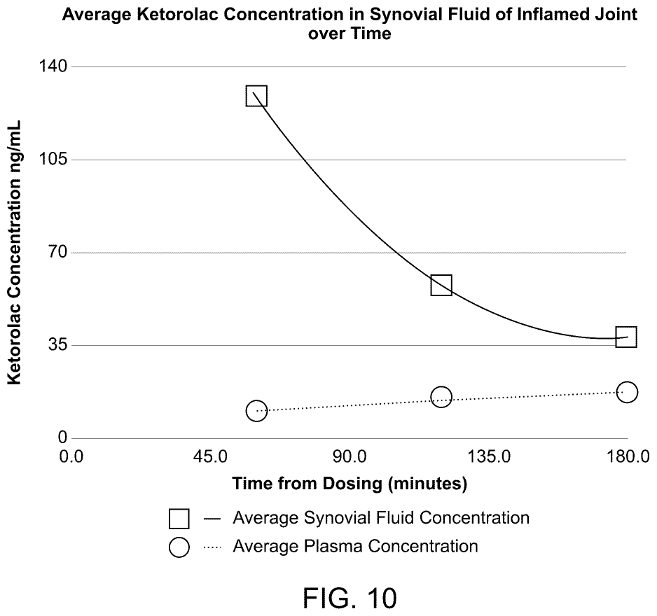

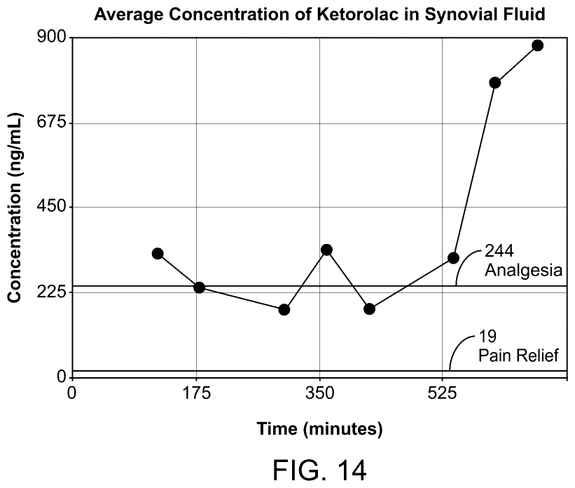

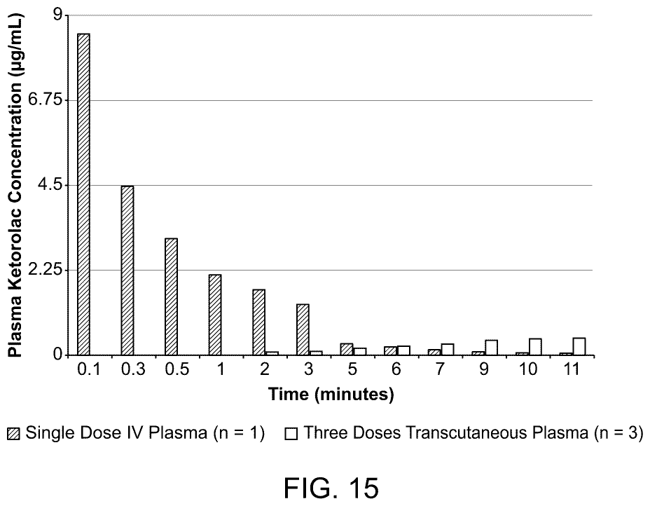

The topical ketorolac compositions also showed delivery of effective drug amounts in an in vivo pig model of gout in single dose and multiple dose experiments. See, Examples 7 and 8, and FIGS. 10-15. In an illustrative study, Composition 125010 delivered pain relief within 60 minutes of administration and pain relief was maintained for at least 11 hours after three times dosing at 4-hour intervals. See, FIGS. 10-13. The multiple dose pig study using a topical ketorolac composition delivered effective amounts of ketorolac to a synovial joint while providing lower plasma ketorolac levels, thus reducing the likelihood of adverse effects of systemic ketorolac exposure. See, FIGS. 14-15.

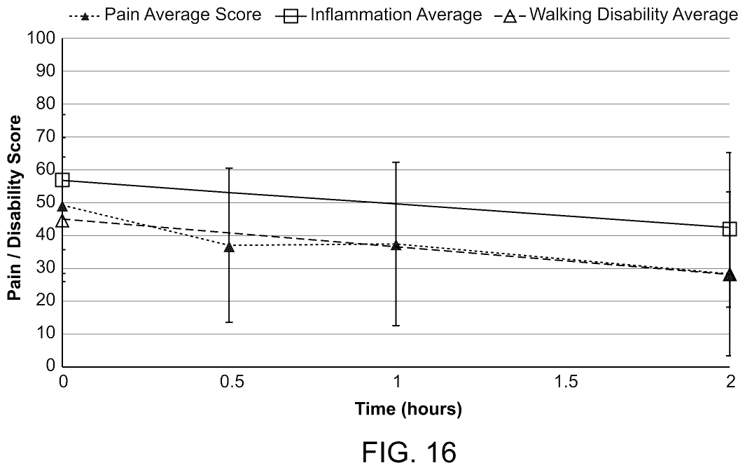

An illustrative topical ketorolac composition 125010 showed phase 1b clinical results that indicated safety as well as efficacy in an open-label trial. See, Example 9 and FIGS. 16-22. No safety or tolerability concerns were observed. No patients used any other medication to control their gout pain during the study. Pain and walking disability improved after a single topical administration of 125010. See, FIG. 16. Based on an average change from baseline, pain (52%), walking disability (44%), and inflammation (21%) levels all demonstrated improvement.

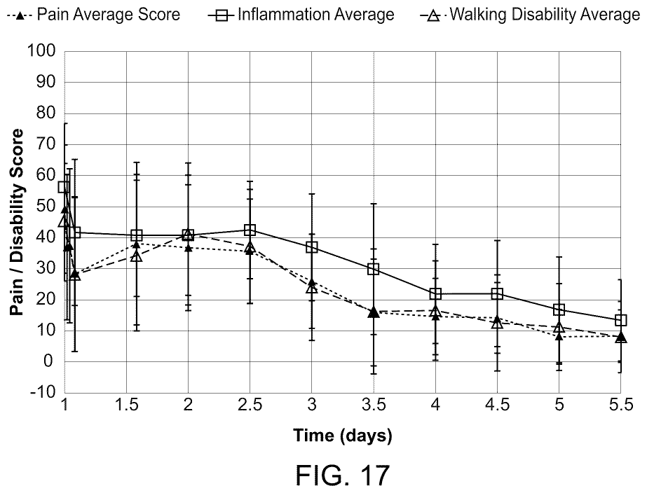

Gout flare symptoms improved over time upon repeated topical administration of 125010 composition. See, FIG. 17. Based on an average change from baseline, pain (71%), walking disability (74%), and inflammation (62%) levels were mild after three days of treatment and nearly resolved after treatment over five days.

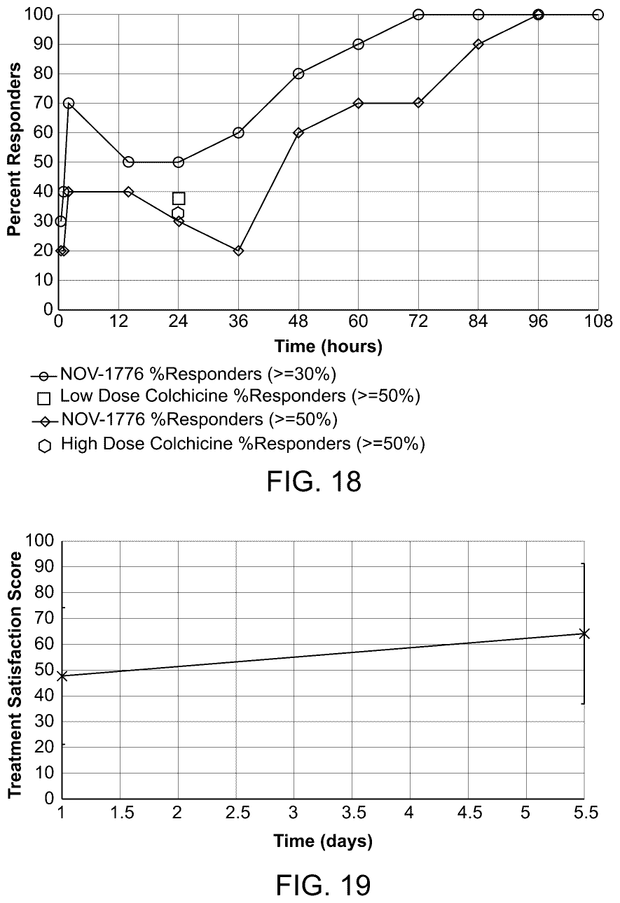

The clinical response rate with 125010 administration was comparable to that reported for colchicine treatment. See, FIG. 18. Three out of ten patients improved by 50% or more by 24 hours after the initial dose of study medication (comparable to what is reported for low dose colchicine (37.8%) and high dose colchicine (32.7%)). See, e.g., Terkeltaub, R. A. et al. Arthritis and Rheumatism, 2010, vol. 62 (4), pages 1060-1068.

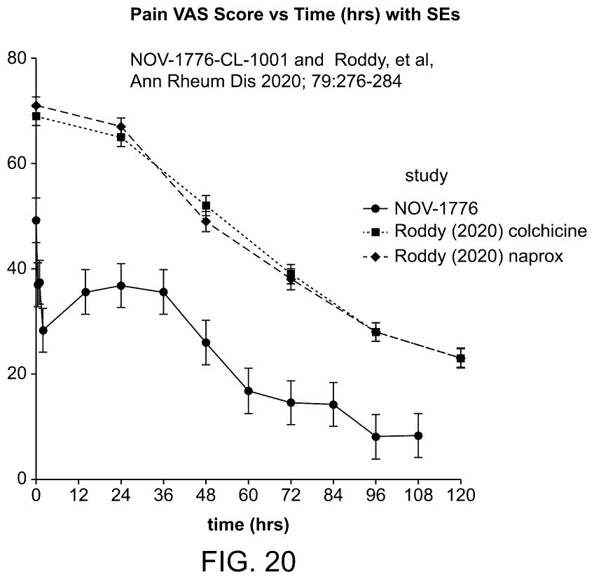

Eight out of 10 participants expressed an improvement in 125010 treatment satisfaction over their prior gout treatment. Five out of 10 participants improved their treatment satisfaction over 80%. See, FIG. 19.

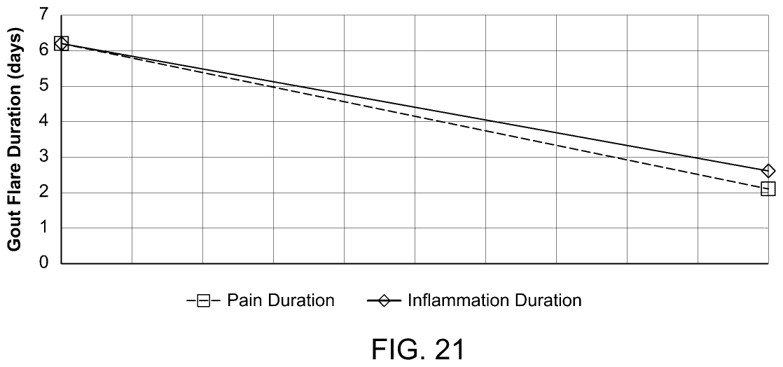

With respect to pain score, 125010 (“NOV-1776”) performed well compared to gout pain standards of care low dose colchicine and naproxen. See, FIG. 20.

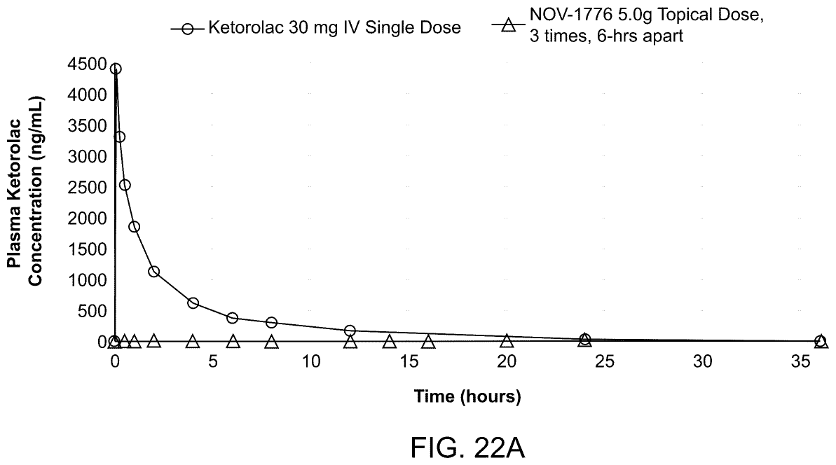

Participants reported reduction in gout flare duration. See, FIG. 21. Days with moderate to severe pain were reduced for 8 out of 10 participants, with an average of 46% duration reduction. Days with moderate to severe inflammation were reduced for 7 out of 10 participants, with an average of 56% duration reduction. At the time of study exit, two participants had moderate swelling, with VAS scores of 34 and 38, respectively.

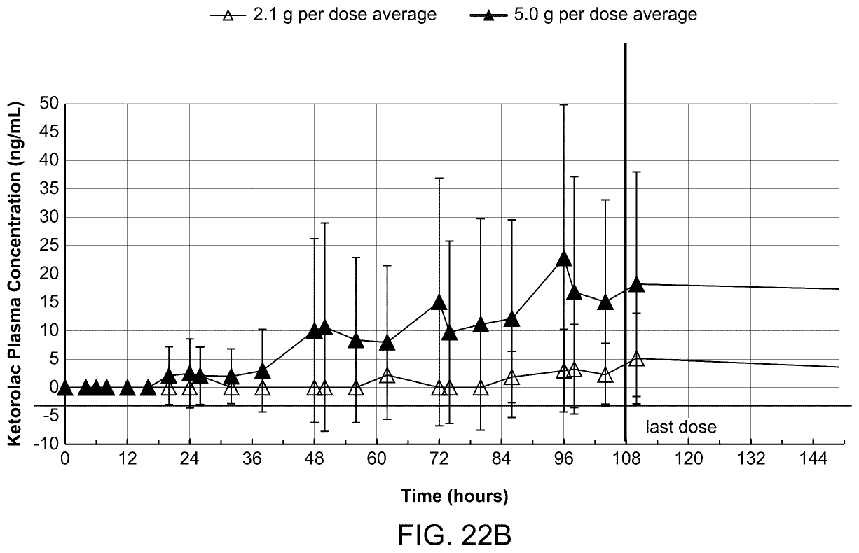

Ketorolac plasma concentrations were significantly lower than that observed for those treated with ketorolac intravenous dosing in a comparative single day dosing regimen. See, FIG. 22A. In repeat dosing over five days, topical administration of 125010 continued to afford low plasma levels of ketorolac with no appreciable accumulation observed. See, FIG. 22B. The low systemic ketorolac exposure suggests that safety concerns for topical administration of the compositions of the present invention would be reduced compared to known ketorolac regimens.

The data in the Examples suggest that the ketorolac compositions of the present invention are capable of delivering a therapeutically effective dose of the active ingredient for daily administration for managing pain and/or inflammation, e.g., joint pain, such as joint pain related to gout.

### II. Definitions

Unless defined otherwise, all technical and scientific terms used herein have the same meaning as commonly understood by one of ordinary skill in the art.

“Pharmaceutically effective amount” refers to an amount of the compound of the present disclosure in a composition or combination thereof, that provides the desired therapeutic or pharmaceutical result.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Treatment” or “treat” or “treating” as used herein refers to an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

“Therapeutically effective amount” or “effective amount” as used herein refers to an amount that is effective to elicit the desired biological or medical response, including the amount of the compound that, when administered to a subject for treating a disease, is sufficient to effect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

“Subject” is any mammal, such as a mouse, a rat, a dog, a cat, including veterinary animals, such as a goat, a pig, a horse, a cow, or a donkey, and primates, such as non-human primates, e.g., a cynomolgous monkey, rhesus monkey, or chimpanzee, as well as humans. In some embodiments, the subject is a human. In some embodiments, a subject is a patient.

### III. Compounds

The present compositions comprise ketorolac or a pharmaceutically acceptable salt thereof. Ketorolac has a chemical structure:

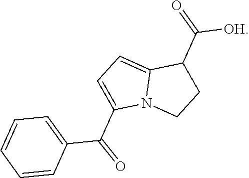 [SMILES: O=C(c1ccccc1)c1ccc2n1CCC2C(=O)O]

In some embodiments, the composition comprises ketorolac tromethamine having structure:

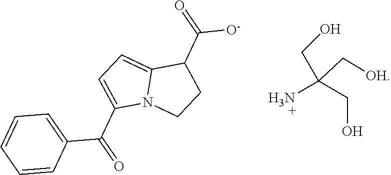 [SMILES: O=C(c1ccccc1)c1ccc2n1CCC2C(=O)[O-] ; NC(CO)(CO)CO]

In some embodiments, the composition comprises choline chloride having structure:

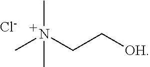

### IV. Compositions

The composition of the present disclosure comprises a eutectic mixture of choline chloride and propylene glycol that serves as a base to incorporate the other ingredients of the topical composition, including the active ingredient ketorolac or pharmaceutically acceptable salt thereof.

As understood in the art, the term “about” means approximately. In some embodiments, the term “about” when referring to a value includes the stated value+/−10% of the stated value. For example, about 50% can include a range of from 45% to 55%, while about 20 molar equivalents can include a range of from 18 to 22 molar equivalents. Accordingly, in some embodiments, when referring to a range, “about” refers to each of the stated values+/−10% of the stated value of each end of the range. For instance, a ratio of from about 1 to about 3 (weight/weight) can include a range of from 0.9 to 3.3.

In some embodiments, a composition of the present disclosure comprises:
(a) from about 1% to about 50% (w/w) ketorolac or pharmaceutically acceptable salt thereof;
(b) from about 5% to about 50% (w/w) choline chloride; and
(c) from about 20% to about 70% (w/w) propylene glycol;
wherein the choline chloride and the propylene glycol form a mixture having a lower melting point than pure propylene glycol.

In some embodiments, a composition of the present disclosure comprises:
(a) from about 1% to about 50% (w/w) ketorolac or pharmaceutically acceptable salt thereof;
(b) from about 5% to about 50% (w/w) choline chloride;
(c) from about 20% to about 70% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, the composition comprises from about 1% to about 40% (w/w), such as from about 1% to about 30%, from about 1% to about 25%, from about 1% to about 20%, from about 1% to about 15%, or from about 2% to about 15% (w/w) ketorolac or pharmaceutically acceptable salt thereof. In some embodiments, the composition comprises about 1%, 1.5%, 2%, 2.1%, 2.2%, 2.3%, 2.4%, 2.5%, 2.6%, 2.7%, 2.8%, 2.9%, 3%, 4%, 5%, 6%, 7%, 8%, 9%, 10%, 11%, 12%, 12.1%, 12.2%, 12.3%, 12.4%, 12.5%, 12.6%, 12.7%, 12.8%, 12.9%, 13%, 14%, 15%, 16%, 17%, 18%, 19%, or about 20% (w/w) ketorolac or pharmaceutically acceptable salt thereof.

In some embodiments of the composition, the ketorolac or pharmaceutically acceptable salt thereof comprises ketorolac tromethamine. In some embodiments, the composition comprises from about 1% to about 40% (w/w), such as from about 1% to about 30%, from about 1% to about 25%, from about 1% to about 20%, from about 1% to about 15%, or from about 2% to about 15% (w/w) ketorolac tromethamine. In some embodiments, the composition comprises about 1%, 1.5%, 2%, 2.1%, 2.2%, 2.3%, 2.4%, 2.5%, 2.6%, 2.7%, 2.8%, 2.9%, 3%, 4%, 5%, 6%, 7%, 8%, 9%, 10%, 11%, 12%, 12.1%, 12.2%, 12.3%, 12.4%, 12.5%, 12.6%, 12.7%, 12.8%, 12.9%, 13%, 14%, 15%, 16%, 17%, 18%, 19%, or about 20% (w/w) ketorolac tromethamine. In some embodiments, the composition comprises about 2.5% (w/w) ketorolac tromethamine. In some embodiments, the composition comprises about 12.5% (w/w) ketorolac tromethamine.

In some embodiments, a composition of the present disclosure comprises:
(a) about 2.5% (w/w) ketorolac or pharmaceutically acceptable salt thereof;
(b) from about 5% to about 50% (w/w) choline chloride;
(c) from about 20% to about 70% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, a composition of the present disclosure comprises:
(a) about 12.5% (w/w) ketorolac or pharmaceutically acceptable salt thereof;
(b) from about 5% to about 50% (w/w) choline chloride;
(c) from about 20% to about 70% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, the composition comprises from about 5% to about 40% (w/w), such as from about 5% to about 35%, from about 5% to about 30%, from about 5% to about 25%, from about 10% to about 30%, from about 15% to about 25%, or from about 18% to about 22% (w/w) choline chloride. In some embodiments, the composition comprises about 5%, 6%, 7%, 8%, 9%, 10%, 11%, 12%, 13%, 14%, 15%, 16%, 17%, 18%, 19%, 19.1%, 19.2%, 19.3%, 19.4%, 19.5%, 19.6%, 19.7%, 19.8%, 19.9%, 20%, 21%, 22%, 23%, 24%, 25%, 26%, 27%, 28%, 29%, or about 30% (w/w) choline chloride. In some embodiments, the composition comprises about 20% (w/w) choline chloride. In some embodiments, the composition comprises about 19.5% (w/w) choline chloride.

In some embodiments, the composition comprises from about 20% to about 60% (w/w), such as from about 20% to about 55%, from about 20% to about 50%, from about 25% to about 50%, from about 25% to about 45%, from about 30% to about 40%, or from about 35% to about 45% (w/w) propylene glycol. In some embodiments, the composition comprises about 25%, 26%, 27%, 28%, 29%, 30%, 31%, 32%, 33%, 34%, 35%, 36%, 37%, 38%, 39%, 39.1%, 39.2%, 39.3%, 39.4%, 39.5%, 39.6%, 39.7%, 39.8%, 39.9%, 40%, 41%, 42%, 43%, 44%, 45%, 46%, 47%, 48%, 49%, or about 50% (w/w) propylene glycol. In some embodiments, the composition comprises about 40% (w/w) propylene glycol. In some embodiments, the composition comprises about 39.4% (w/w) propylene glycol.

In some embodiments of the composition, the choline chloride and the propylene glycol is present in an amount of from about 5:2 to about 1:10, such as from about 5:2 to about 1:5, from about 2:1 to about 1:5, from about 2:1 to about 1:4, from about 1:1 to about 1:5, from about 1:1 to about 1:4, or from about 1:1 to about 1:3 by weight. In some embodiments of the composition, the choline chloride and the propylene glycol is present in an amount of about 5:2, 2:1, 5:3, 3:2, 1:1, 1:2, 1:3, 1:4, or about 1:5 by weight. In some embodiments of the composition, the choline chloride and the propylene glycol is present in an amount of about 2:1.

In some embodiments, a composition of the present disclosure comprises:
(a) from about 2% to about 30% (w/w) ketorolac tromethamine;
(b) from about 15% to about 25% (w/w) choline chloride;
(c) from about 20% to about 60% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, a composition of the present disclosure comprises:
(a) from about 2% to about 20% (w/w) ketorolac tromethamine;
(b) from about 15% to about 25% (w/w) choline chloride;
(c) from about 20% to about 50% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, a composition of the present disclosure comprises:
(a) about 2.5% (w/w) ketorolac tromethamine;
(b) from about 5% to about 40% (w/w) choline chloride;
(c) from about 20% to about 50% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, a composition of the present disclosure comprises:
(a) about 12.5% (w/w) ketorolac tromethamine;
(b) from about 5% to about 40% (w/w) choline chloride;
(c) from about 20% to about 50% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, a composition of the present disclosure comprises:
(a) about 2.5% (w/w) ketorolac tromethamine;
(b) from about 10% to about 40% (w/w) choline chloride;
(c) from about 20% to about 70% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, a composition of the present disclosure comprises:
(a) about 12.5% (w/w) ketorolac tromethamine;
(b) from about 10% to about 40% (w/w) choline chloride;
(c) from about 20% to about 70% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, a composition of the present disclosure comprises:
(a) about 2.5% (w/w) ketorolac tromethamine;
(b) from about 15% to about 25% (w/w) choline chloride;
(c) from about 20% to about 70% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, a composition of the present disclosure comprises:
(a) about 12.5% (w/w) ketorolac tromethamine;
(b) from about 15% to about 25% (w/w) choline chloride;
(c) from about 20% to about 70% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, a composition of the present disclosure comprises:
(a) from about 2% to about 30% (w/w) ketorolac tromethamine;
(b) about 20% (w/w) choline chloride;
(c) from about 20% to about 70% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, a composition of the present disclosure comprises:
(a) from about 2% to about 20% (w/w) ketorolac tromethamine;
(b) about 20% (w/w) choline chloride;
(c) from about 20% to about 70% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, a composition of the present disclosure comprises:
(a) from about 2% to about 30% (w/w) ketorolac tromethamine;
(b) from about 15% to about 25% (w/w) choline chloride;
(c) about 40% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, a composition of the present disclosure comprises:
(a) from about 2% to about 20% (w/w) ketorolac tromethamine;
(b) from about 15% to about 25% (w/w) choline chloride;
(c) about 40% (w/w) propylene glycol; and
(d) from about 10% to about 30% (w/w) dimethyl sulfoxide;
wherein the choline chloride and the propylene glycol form a mixture having a lower melting point than pure propylene glycol.

In some embodiments, a composition of the present disclosure comprises:
(a) from about 2% to about 30% (w/w) ketorolac tromethamine;
(b) from about 15% to about 25% (w/w) choline chloride;
(c) from about 20% to about 60% (w/w) propylene glycol; and
(d) about 20% (w/w) dimethyl sulfoxide;
wherein the choline chloride and the propylene glycol form a mixture having a lower melting point than pure propylene glycol.

In some embodiments, a composition of the present disclosure comprises:
(a) from about 2% to about 20% (w/w) ketorolac tromethamine;
(b) from about 15% to about 25% (w/w) choline chloride;
(c) from about 20% to about 50% (w/w) propylene glycol; and
(d) about 20% (w/w) dimethyl sulfoxide;
wherein the choline chloride and the propylene glycol form a mixture having a lower melting point than pure propylene glycol.

In some embodiments, the composition comprises from about 10% to about 30% (w/w), such as from about 10% to about 25%, from about 15% to about 30%, from about 15% to about 25%, or from about 18% to about 22% (w/w) dimethyl sulfoxide. In some embodiments, the composition comprises about 10%, 11%, 12%, 13%, 14%, 15%, 16%, 17%, 18%, 19%, 19.1%, 19.2%, 19.3%, 19.4%, 19.5%, 19.6%, 19.7%, 19.8%, 19.9%, 20%, 21%, 22%, 23%, 24%, 25%, 26%, 27%, 28%, 29%, or about 30% (w/w) dimethyl sulfoxide. In some embodiments, the composition comprises about 20% (w/w) dimethyl sulfoxide. In some embodiments, the composition comprises about 19.6% (w/w) dimethyl sulfoxide.

In some embodiments, the composition comprises:
(a) from about 10% to about 15% (w/w) ketorolac tromethamine;
(b) from about 15% to about 25% (w/w) choline chloride;
(c) from about 35% to about 45% (w/w) propylene glycol; and
(d) from about 15% to about 25% (w/w) dimethyl sulfoxide.

In some embodiments, the composition comprises:
(a) from about 10% to about 15% (w/w) ketorolac tromethamine;
(b) from about 20% to about 25% (w/w) choline chloride;
(c) from about 40% to about 50% (w/w) propylene glycol; and
(d) from about 15% to about 25% (w/w) dimethyl sulfoxide.

In some embodiments, the composition comprises:
(a) about 12.5% (w/w) ketorolac tromethamine;
(b) about 20% (w/w) choline chloride;
(c) about 40% (w/w) propylene glycol; and
(d) about 20% (w/w) dimethyl sulfoxide.

In some embodiments, the composition comprises:
(a) about 12.5% (w/w) ketorolac tromethamine;
(b) about 23% (w/w) choline chloride;
(c) about 45% (w/w) propylene glycol; and
(d) about 20% (w/w) dimethyl sulfoxide.

In some embodiments, the composition comprises:
(a) about 12.5% (w/w) ketorolac tromethamine;
(b) about 19.5% (w/w) choline chloride;
(c) about 39.4% (w/w) propylene glycol; and
(d) about 19.6% (w/w) dimethyl sulfoxide.

Compositions provided herein may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

In some embodiments, the composition further comprises an antioxidant. Any antioxidant known in topical compositions can be used in the present compositions. In some embodiments, the antioxidant is vitamin E (e.g., α-tocopherol), coenzyme Q10, butylated hydroxytoluene (BHT), butylated hydroxyanisole (BTA), propyl gallate, ethylenediaminetetraacetic (EDTA), sodium bicarbonate, idebenone, lycopene, ascorbic acid or ester thereof, e.g., ascorbyl palmitate, a green tea polyphenol, e.g., epigallocatechin 3-gallate (EGCG), a polyphenolic flavonoid, e.g., silymarin, resveratrol, genistein, pycnogenol, niacinamide, or combinations thereof.

In some embodiments, the antioxidant is present in an amount of from about 0.01% to about 1% by weight. In some embodiments, the antioxidant is present in an amount of from about 0.01% to about 0.5% by weight. In some embodiments, the antioxidant is present in an amount of from about 0.01% to about 0.1% by weight. In some embodiments, the antioxidant is present in an amount of from about 0.1% to about 0.5% by weight. In some embodiments, the antioxidant is present in an amount of from about 0.1% to about 0.3% by weight.

In some embodiments, the composition further comprises an emulsifier. Emulsifiers are added to creams and lotions to form homogenous mixtures of both. Addition of an emulsifier not only stabilizes the emulsion but also influences the consistency of the composition. Emulsifiers are used in creams, lotions, sprays and foams and are categorized as oil in water or water in oil. Oil in water emulsifiers keep oil drops packed in water and are used more in moisturizing products; water in oil emulsifiers keep water droplets packed in oil and are used for a fatty feel. Emulsifiers include lecithin, glyceryl stearate and cetearyl alcohol.

In some embodiments, the composition further comprises glyceryl stearate. In some embodiments, the composition comprises from about 0.5% to about 10% (w/w) glyceryl stearate. In some embodiments, the composition comprises from about 5% to about 10% (w/w) glyceryl stearate. In some embodiments, the composition comprises about 0.5%, 0.6%, 0.7%, 0.8%, 0.9%, 1%, 2%, 3%, 4%, 5%, 6%, 7%, 8%, 9%, or about 10% (w/w) glyceryl stearate. In some embodiments, the composition comprises about 7% (w/w) glyceryl stearate.

In some embodiments, the emulsifier comprises a surfactant. In some embodiments, the emulsifier comprises polyethylene glycol monododecyl ether.

Span® 80, also known as sorbitan monooleate, is a sorbitan ester series of surfactant which is produced by esterification of one or more sorbitan hydroxyl group with fatty acid.

In some embodiments, the composition further comprises sorbitan monooleate. In some embodiments, the composition comprises from about 0.01% to about 1% (w/w) sorbitan monooleate. In some embodiments, the composition comprises from about 0.1% to about 0.5% (w/w) sorbitan monooleate. In some embodiments, the composition comprises about 0.01%. 0.05%, 0.1%, 0.15%, 0.2%, 0.25%, 0.3%, 0.4%, 0.5%, 0.6%, 0.7%, 0.8%, 0.9%, or about 1% (w/w) sorbitan monooleate. In some embodiments, the composition comprises about 0.2% (w/w) sorbitan monooleate.

Polysorbate 80 (TWEEN 80) is a common fragrance and essential oil solubilizer as well as an emulsifier. It is soluble in water and alcohol and insoluble in oils. It is typically a co-emulsifier that can be used both as a viscosity modifier and a dispersing agent.

In some embodiments, the composition further comprises polysorbate 80. In some embodiments, the composition comprises from about 0.5% to about 3% (w/w) polysorbate 80. In some embodiments, the composition comprises from about 1% to about 3% (w/w) polysorbate 80. In some embodiments, the composition comprises about 0.5%, 0.6%, 0.7%, 0.8%, 0.9%, 1%, 1.1%, 1.2%, 1.3%, 1.4%, 1.5%, 1.6%, 1.7%, 1.8%, 1.9%, or about 2% (w/w) polysorbate 80. In some embodiments, the composition comprises about 1.8% (w/w) polysorbate 80.

In some embodiments, the composition comprises:
(a) from about 10% to about 15% (w/w) ketorolac tromethamine;
(b) from about 15% to about 25% (w/w) choline chloride;
(c) from about 35% to about 45% (w/w) propylene glycol;
(d) from about 15% to about 25% (w/w) dimethyl sulfoxide; and
(e) from about 5% to about 10% (w/w) glyceryl stearate.

In some embodiments, the composition comprises:
(a) from about 10% to about 15% (w/w) ketorolac tromethamine;
(b) from about 20% to about 25% (w/w) choline chloride;
(c) from about 40% to about 50% (w/w) propylene glycol;
(d) from about 15% to about 25% (w/w) dimethyl sulfoxide; and
(e) from about 5% to about 10% (w/w) glyceryl stearate.

In some embodiments, the composition comprises:
(a) from about 10% to about 15% (w/w) ketorolac tromethamine;
(b) from about 15% to about 25% (w/w) choline chloride;
(c) from about 35% to about 45% (w/w) propylene glycol;
(d) from about 15% to about 25% (w/w) dimethyl sulfoxide;
(e) from about 5% to about 10% (w/w) glyceryl stearate; and
(f) from about 1% to about 3% (w/w) polysorbate 80.

In some embodiments, the composition comprises:
(a) from about 10% to about 15% (w/w) ketorolac tromethamine;
(b) from about 20% to about 25% (w/w) choline chloride;
(c) from about 40% to about 50% (w/w) propylene glycol;
(d) from about 15% to about 25% (w/w) dimethyl sulfoxide;
(e) from about 5% to about 10% (w/w) glyceryl stearate; and
(f) from about 1% to about 3% (w/w) polysorbate 80.

In some embodiments, the composition comprises:
(a) about 12.5% (w/w) ketorolac tromethamine;
(b) about 19.5% (w/w) choline chloride;
(c) about 39.4% (w/w) propylene glycol;
(d) about 19.6% (w/w) dimethyl sulfoxide;
(e) about 7.0% (w/w) glyceryl stearate;
(f) about 0.2% (w/w) sorbitan monooleate; and
(g) about 1.8% (w/w) polysorbate 80.

Exemplary compositions of the present disclosure include those in the tables below.

| | 125001 | 25001 | 25002 | 25003 | 25004 | 25010* | 25011^{§} | 25012 | “B” | “C” |
|---|---|---|---|---|---|---|---|---|---|---|
| ketorolac | 12.5 | 2.5 | 2.5 | 2.5 | 2.5 | 2.5 | 2.5 | 2.5 | 3.75 | 7.5 |
| tromethamine | | | | | | | | | | |
| choline | 22.52 | 33.45 | 26 | 36.8 | 43.39 | 6.062 | — | 20.8 | 36.84 | 35.4 |
| chloride | | | | | | | | | | |
| propylene | 45.41 | 36.57 | 52 | 30.1 | 23.49 | 26.975 | — | 41.6 | 40.16 | 38.59 |
| glycol | | | | | | | | | | |
| propylene | — | — | — | — | — | 12.932 | — | — | — | — |
| glycol | | | | | | | | | | |
| slurry | | | | | | | | | | |
| dimethyl | 19.58 | 17.58 | 19.58 | 17.58 | 17.58 | — | — | 15.6 | 19.25 | 18.5 |
| sulfoxide | | | | | | | | | | |
| Laureth- | — | 10 | — | 10 | 10 | — | 20.5 | 19.5 | — | — |
| 23 | | | | | | | | | | |

*Composition 25010 also includes 3.917% glycerol monosterate, 3.917% cetyl alcohol, 2.549% oleic acid, 2.549% polysorbate 20, 3.917% squalene, 1.293% dimethacone, 1.293% xanthan gum, and 32.094% water.
^{§}Composition 25011 also includes 77.5% water.

| | 125002 | 125003 | 125004 | 125010^{§} | 25013^{¥} |
|---|---|---|---|---|---|
| ketorolac | 12.5 | 12.5 | 12.5 | 12.5 | 2.5 |
| tromethamine | | | | | |
| choline | 20.9 | 25.7 | 24.0 | 19.5 | 13.448 |
| chloride | | | | | |
| propylene | 42.1 | 51.8 | 48.5 | 39.4 | 14.658 |
| glycol | | | | | |
| dimethyl | 19.6 | 10 | 10 | 19.6 | — |
| sulfoxide | | | | | |
| Laureth-23 | 5 | 0 | 5 | 0 | 5.621 |

^{§}Composition 125010 also included 7.0% glyceryl monostearate, 0.2% SPAN 80 (surfactant for homogenizing gel) and 1.8% polysorbate 80 (surfactant for homogenizing gel).
^{¥}Composition 25013 also included 5.621% glyceryl monosterate, 5.621% cetyl alcohol, 3.373% oleic acid, 5.621% polysorbate 20, 5.621% squalene, 6.745% dimethacone, 0.787% xanthan gum, 3.373% isopropyl myristate, and 28.106% water.

| | 125010 | 125011 | 125012 | 125013 | 125016 | 125018 |
|---|---|---|---|---|---|---|
| ketorolac | 12.5 | 12.5 | 12.5 | 12.5 | 12.5 | 12.5 |
| tromethamine | | | | | | |
| choline | 19.5 | 0 | 22.5 | 29.0 | 38.1 | 9.1 |
| chloride | | | | | | |
| propylene | 39.4 | 0 | 45.4 | 58.5 | 20.8 | 49.8 |
| glycol | | | | | | |
| dimethyl | 19.6 | 78.5 | 19.6 | 0 | 19.6 | 19.6 |
| sulfoxide | | | | | | |
| glyceryl | 7 | 7 | 0 | 0 | 7 | 7 |
| stearate | | | | | | |
| sorbitan oleate | 0.18 | 0.18 | 0 | 0 | 0.18 | 0.18 |
| (SPAN 80) | | | | | | |
| polysorbate 80 | 1.8 | 1.8 | 0 | 0 | 1.8 | 1.8 |

### Preparation

The compositions of the present disclosure can be prepared by any method known in the art for preparing compositions containing eutectic mixtures. Typically, the eutectic solvent can be first prepared by heating choline chloride and propylene glycol to form a uniform solution, followed by addition of a mixture containing ketorolac or pharmaceutically acceptable salt thereof, e.g., ketorolac tromethamine, to prepare the composition. In some embodiments, the eutectic mixture is prepared by mixing the choline chloride and propylene glycol at elevated temperature, e.g., from about 50° C. to about 80° C., such as from about 60° C. to about 75° C., e.g., at about 50, 55, 60, 65, 70, or about 75° C., before combining with the ketorolac or pharmaceutically acceptable salt thereof. Accordingly, in some embodiments, the choline chloride and the propylene glycol in the present composition form a mixture having a lower melting point of from about 0.01° C. to about 10° C., such as from about 0.01° C. to about 8° C., about 0.01° C. to about 6° C., about 0.01° C. to about 5° C., about 0.01° C. to about 4° C., about 0.01° C. to about 3° C., about 0.01° C. to about 2° C., about 0.01° C. to about 1° C., as compared to pure propylene glycol. In some embodiments, the choline chloride and the propylene glycol in the present composition form a mixture having a lower melting point of about 0.01° C., 0.02° C., 0.03° C., 0.04° C., 0.05° C., 0.06° C., 0.07° C., 0.08° C., 0.09° C., 0.1° C., 0.2° C., 0.3° C., 0.4° C., 0.5° C., 0.6° C., 0.7° C., 0.8° C., 0.9° C., 1° C., 2° C., 3° C., 4° C., 5° C., 6° C., 7° C., 8° C., 9° C., or about 10° C. as compared to pure propylene glycol.

In some embodiments, the mixture containing ketorolac or pharmaceutically acceptable salt thereof comprises other ingredients, such as a surfactant, e.g., polysorbate 80, and/or an emulsifier, e.g., glyceryl stearate. In some embodiments, the mixture containing ketorolac or pharmaceutically acceptable salt thereof comprises one or more of dimethyl sulfoxide, glyceryl stearate, sorbitan oleate, and polysorbate 80.

In some embodiments, the mixture containing ketorolac or pharmaceutically acceptable salt thereof is added to the eutectic mixture at elevated temperature, e.g., from about 50° C. to about 80° C., such as from about 60° C. to about 75° C., e.g., at about 50, 55, 60, 65, 70, or about 75° C., to prepare the composition of the disclosure.

### Administration

The compositions that comprise ketorolac or a pharmaceutically acceptable salt thereof can be administered by any useful route and means, such as by topical administration, in various forms including but not limited to a liquid, patch, cream, lotion, or gel. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 mg to about 30 mg per day, or such as from about 30 mg to about 300 mg per day.

In some embodiments, the composition of the present invention is administered in about 0.6 g, about 2.1 g, or about 5.0 g per dose. In some embodiments, the composition of the present invention is administered in about 0.6 g per dose. In some embodiments, the composition of the present invention is administered in about 2.1 g per dose. In some embodiments, the composition of the present invention is administered in about 5 g per dose.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel compositions (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

Lipid-based drug delivery systems include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the compositions of the present invention include any natural or synthetic lipids including, but not limited to, sesame seed oil, olive oil, castor oil, peanut oil, fatty acid esters, glycerol esters, Labrafil®, Labrasol®, Cremophor®, Solutol®, Tween®, Capryol®, Capmul®, Captex®, and Peceol®.

### V. Methods

The present disclosure provides methods and uses for treating inflammation and/or pain comprising topically administering ketorolac compositions. The methods and uses described herein provide an effective amount of the ketorolac while reducing the incidence of one or more of the adverse effects observed with treatment by ketorolac administration by other means, such as intravenous, intramuscular, or oral administration.

In some embodiments, the use of the present disclosure is a use for the manufacture of a medicament in a method of treating inflammation and/or pain as described herein.

Ketorolac tromethamine is marketed as Toradol (Roche). Adverse effects of ketorolac tromethamine include gastrointestinal (GI) effects, such as abdominal pain, constipation, diarrhea, dyspepsia, flatulence, GI fullness, GI ulcers (including gastric and duodenal ulcers), stomatitis, and vomiting; abnormal renal function; anemia; dizziness; drowsiness; edema; elevated liver enzymes; headaches; hypertension; increased bleeding time; pruritus; purpura; rashes; tinnitus; and sweating.

In some embodiments, the method or use of the present disclosure reduces the incidence and/or severity of one or more of the adverse effects in a subject by from about 10% to about 90%, such as from about 20% to about 90%, from about 20% to about 80%, from about 20% to about 70%, from about 20% to about 60%, from about 20% to about 50%, from about 20% to about 40%, from about 30% to about 90%, from about 30% to about 80%, from about 30% to about 70%, from about 30% to about 60%, or from about 30% to about 50% as compared to the incidence and/or severity of one or more of the adverse effects in the subject treated with oral ketorolac tromethamine. In some embodiments, the method or use of the present disclosure reduces the incidence and/or severity of one or more of the adverse effects in a subject by about 10%, 20%, 30%, 40%, 50%, 60%, 70%, 80%, or about 90% as compared to the incidence and/or severity of one or more of the adverse effects in the subject treated with oral ketorolac tromethamine.

In some embodiments, the method or use of the present disclosure reduces the probability of one or more of the adverse effects in a subject by from about 10% to about 90%, such as from about 20% to about 90%, from about 20% to about 80%, from about 20% to about 70%, from about 20% to about 60%, from about 20% to about 50%, from about 20% to about 40%, from about 30% to about 90%, from about 30% to about 80%, from about 30% to about 70%, from about 30% to about 60%, or from about 30% to about 50% as compared to the probability of one or more of the adverse effects in the subject treated with oral ketorolac tromethamine. In some embodiments, the method or use of the present disclosure reduces the probability of one or more of the adverse effects in a subject by about 10%, 20%, 30%, 40%, 50%, 60%, 70%, 80%, or about 90% as compared to the probability of one or more of the adverse effects in the subject treated with oral ketorolac tromethamine.

In some embodiments, the method of the present disclosure is a method of treating inflammation or pain in a subject in need thereof, comprising topically administering to the subject the composition of the present disclosure. In some embodiments, the method of the present disclosure is a method of treating inflammation or pain associated with acute gout in a human subject in need thereof, comprising topically administering to a joint in the subject a therapeutically effective amount of the composition of the present disclosure.

In some embodiments, the method of the present disclosure is a method of treating inflammation or pain associated with acute gout in a human subject in need thereof, comprising topically administering three times daily to a joint in the subject a therapeutically effective amount of the composition of the present disclosure. In some embodiments, the composition is administered in about 5.0 g per dose. In some embodiments, the composition comprises: (a) about 12.5% (w/w) ketorolac tromethamine; (b) about 19.5% (w/w) choline chloride; (c) about 39.4% (w/w) propylene glycol; (d) about 19.6% (w/w) dimethyl sulfoxide; (e) about 7.0% (w/w) glyceryl stearate; (f) about 0.2% (w/w) sorbitan monooleate; and (g) about 1.8% (w/w) polysorbate 80. In some embodiments, the composition is administered for at least about 1 day, at least about 2 days, at least about 3 days, at least about 4 days, or for at least about 5 days.

Without being bound by theory, the methods and uses of the present disclosure are believed to deliver locally, e.g., to a joint, a therapeutically effective amount of ketorolac to a subject in need thereof while reducing the systemic, e.g., plasma, ketorolac levels in the subject. In some embodiments, the method or use of the present disclosure affords a low level of ketorolac, e.g., less than about 100 μg/mL, such as less than about 50 μg/mL, less than about 40 μg/mL, less than about 30 μg/mL, less than about 20 μg/mL, less than about 10 μg/mL, less than about 9 μg/mL, less than about 8 μg/mL, less than about 7 μg/mL, less than about 6 μg/mL, less than about 5 μg/mL, less than about 4 μg/mL, less than about 3 μg/mL, less than about 2 μg/mL, less than about 1 μg/mL, less than about 0.9 μg/mL, less than about 0.8 μg/mL, less than about 0.7 μg/mL, less than about 0.6 μg/mL, less than about 0.5 μg/mL, less than about 0.4 μg/mL, less than about 0.3 μg/mL, less than about 0.2 μg/mL, less than about 0.1 μg/mL, less than about 90 ng/mL, less than about 80 ng/mL, less than about 70 ng/mL, less than about 60 ng/mL, less than about 50 ng/mL, less than about 40 ng/mL, less than about 30 ng/ml, less than about 20 ng/ml, or less than about 10 ng/mL ketorolac as measured in plasma from a subject after topical administration of a composition of the present disclosure. In some embodiments, less than about 1 μg/mL, less than about 0.1 μg/mL, or less than about 10 ng/mL ketorolac is measured in plasma from a subject after topical administration of a composition of the present disclosure. In some embodiments, less than about 0.1 μg/mL, less than about 90 ng/ml, less than about 80 ng/mL, less than about 70 ng/mL, less than about 60 ng/ml, less than about 50 ng/ml, less than about 40 ng/mL, less than about 30 ng/ml, less than about 20 ng/ml, or less than about 10 ng/mL ketorolac is measured in plasma from a subject after topical administration of a composition of the present disclosure. In some embodiments, less than about 0.1 μg/mL ketorolac is measured in plasma from a subject after topical administration of a composition of the present disclosure. In some embodiments, less than about 90 ng/mL ketorolac is measured in plasma from a subject after topical administration of a composition of the present disclosure. In some embodiments, less than about 80 ng/mL ketorolac is measured in plasma from a subject after topical administration of a composition of the present disclosure. In some embodiments, less than about 70 ng/mL ketorolac is measured in plasma from a subject after topical administration of a composition of the present disclosure. In some embodiments, less than about 60 ng/mL ketorolac is measured in plasma from a subject after topical administration of a composition of the present disclosure. In some embodiments, less than about 50 ng/mL ketorolac is measured in plasma from a subject after topical administration of a composition of the present disclosure. In some embodiments, less than about 40 ng/mL ketorolac is measured in plasma from a subject after topical administration of a composition of the present disclosure. In some embodiments, less than about 30 ng/mL ketorolac is measured in plasma from a subject after topical administration of a composition of the present disclosure. In some embodiments, less than about 20 ng/mL ketorolac is measured in plasma from a subject after topical administration of a composition of the present disclosure. In some embodiments, less than about 10 ng/ml ketorolac is measured in plasma from a subject after topical administration of a composition of the present disclosure.

In some embodiments, the low systemic level of ketorolac, e.g., in the plasma, is measured soon after administration of the topical administration of the composition of the present disclosure. In some embodiments, the ketorolac is measured in plasma from a subject about 5 min, about 10 min, about 15 min, about 20 min, about 25 min, about 30 min, about 35 min, about 40 min, about 45 min, about 50 min, about 55 min, about 1 hr, about 2 hr, about 3 hr, about 4 hr, about 5 hr, about 6 hr, about 7 hr, about 8 hr, about 9 hr, about 10 hr, about 1 hr, or about 12 hr, after topical administration of a composition of the present disclosure. In some embodiments, the ketorolac is measured in plasma from a subject immediately after, e.g., about 5 min, about 10 min, about 15 min, after topical administration of a composition of the present disclosure.

In some embodiments, the method comprises treating inflammation. In some embodiments, the inflammation comprises an inflammation of the joint. In some embodiments, the inflammation comprises gout.

Gout generally refers to a disorder or condition associated with the buildup of uric acid, such as deposition of uric crystals in tissues and joints, and/or a clinically relevant elevated serum uric acid level. Accumulation of uric acid may be due to overproduction of uric acid or reduced excretion of uric acid. Gout may range from asymptomatic to severe and painful inflammatory conditions. A condition associated with gout refers to any condition in a subject where the subject experiences local and/or systemic effects of gout, including inflammation and immune responses, and in which the condition is caused or exacerbated by, or the condition can result in or exacerbate, gout. A gout flare is an attack or exacerbation of gout symptoms, which can happen at any time. Gout flares can include gout flares that occur after the administration of a uric acid lowering therapy.

In some embodiments, the subject has or is at risk of having an elevated uric acid level, e.g., an elevated plasma or serum uric acid level. When blood levels of uric acid may exceed the physiologic limit of solubility, the uric acid may crystallize in the tissues, including the joints, and may cause gout and gout-associated conditions.

In some embodiments, serum uric acid levels ≥5 mg/dL, ≥6 mg/dL, or ≥7 mg/dl are indicative that a subject may be a candidate for treatment with any one of the methods or compositions or kits described herein. In some embodiments, such a subject has a serum level of uric acid≥6 mg/dL, for example, between 6.1 mg/dL-15 mg/dL, between 6.1 mg/dL-10 mg/dL, 7 mg/dL-15 mg/dL, 7 mg/dL-10 mg/dL, 8 mg/dL-15 mg/dL, 8 mg/dL-10 mg/dL, 9 mg/dL-15 mg/dL, 9 mg/dL-10 mg/dL, 10 mg/dL-15 mg/dL, or 11 mg/dL-14 mg/dL. In some embodiments, the subject has serum level of uric acid of about 6.1 mg/dL, 6.2 mg/dL, 6.3 mg/dL, 6.4 mg/dL, 6.5 mg/dL, 6.7 mg/dL, 6.8 mg/dL, 6.9 mg/dL, 7.0 mg/dL, 7.1 mg/dL, 7.2 mg/dL, 7.3 mg/dL, 7.4 mg/dL, 7.5 mg/dL, 7.6 mg/dL 7.7 mg/dL, 7.8 mg/dL, 7.9 mg/dL, 8.0 mg/dL, 8.1 mg/dL, 8.2 mg/dL, 8.3 mg/dL, 8.4 mg/dL, 8.5 mg/dL, 8.6 mg/dL, 8.7 mg/dL, 8.8 mg/dL, 8.9 mg/dL, 9.0 mg/dL, 9.1 mg/dL, 9.2 mg/dL, 9.3 mg/dL, 9.4 mg/dL, 9.5 mg/dL, 9.6 mg/dL, 9.7 mg/dL, 9.8 mg/dL, 9.9 mg/dL, 10.0 mg/dL, 10.1 mg/dL, 10.2 mg/dL, 10.3 mg/dL, 10.4 mg/dL, 10.5 mg/dL, 10.6 mg/dL, 10.7 mg/dL, 10.8 mg/dL, 10.9 mg/dL, 11.0 mg/dL, 11.1 mg/dL, 11.2 mg/dL, 11.3 mg/dL, 11.4 mg/dL, 11.5 mg/dL, 11.6 mg/dL, 11.7 mg/dL, 11.8 mg/dL, 11.9 mg/dL, 12.0 mg/dL, 12.1 mg/dL, 12.2 mg/dL, 12.3 mg/dL, 12.4 mg/dL, 12.5 mg/dL, 12.6 mg/dL, 12.7 mg/dL, 12.8 mg/dL, 12.9 mg/dL, 13.0 mg/dL, 13.1 mg/dL, 13.2 mg/dL, 13.3 mg/dL, 13.4 mg/dL, 13.5 mg/dL, 13.6 mg/dL, 13.7 mg/dL, 13.8 mg/dL, 13.9 mg/dL, 14.0 mg/dL, 14.1 mg/dL, 14.2 mg/dL, 14.3 mg/dL, 14.4 mg/dL, 14.5 mg/dL, 14.6 mg/dL, 14.7 mg/dL, 14.8 mg/dL, 14.9 mg/dL, 15.0 mg/dL or higher. In some embodiments, the subject has a plasma or serum uric acid level of 5.0 mg/dL, 5.1 mg/dL, 5.2 mg/dL, 5.3 mg/dL, 5.4 mg/dL, 5.5 mg/dL, 5.6 mg/dL, 5.7 mg/dL, 5.8 mg/dL, 5.9 mg/dL, 6.0 mg/dL, 6.1 mg/dL, 6.2 mg/dL, 6.3 mg/dL, 6.4 mg/dL, 6.5 mg/dL, 6.6 mg/dL, 6.7 mg/dL, 6.8 mg/dL, 6.9 mg/dL, or 7.0 mg/dL. In some embodiments, the subject has a plasma or serum uric acid level of greater than or equal to 5.0 mg/dL, 5.1 mg/dL, 5.2 mg/dL, 5.3 mg/dL, 5.4 mg/dL, 5.5 mg/dL, 5.6 mg/dL, 5.7 mg/dL, 5.8 mg/dL, 5.9 mg/dL, 6.0 mg/dL, 6.1 mg/dL, 6.2 mg/dL, 6.3 mg/dL, 6.4 mg/dL, 6.5 mg/dL, 6.6 mg/dL, 6.7 mg/dL, 6.8 mg/dL, 6.9 mg/dL, or 7.0 mg/dL.

In some embodiments, the method or use of the present disclosure reduces the plasma or serum uric acid level of a subject by from about 10% to about 90%, such as from about 20% to about 90%, from about 20% to about 80%, from about 20% to about 70%, from about 20% to about 60%, from about 20% to about 50%, from about 20% to about 40%, from about 30% to about 90%, from about 30% to about 80%, from about 30% to about 70%, from about 30% to about 60%, or from about 30% to about 50% as compared to the initial plasma or serum uric acid level of the subject before treatment with a composition of the present disclosure. In some embodiments, the method or use of the present disclosure reduces the plasma or serum uric acid level of a subject by about 10%, 20%, 30%, 40%, 50%, 60%, 70%, 80%, or about 90% as compared to the initial plasma or serum uric acid level of the subject before treatment with a composition of the present disclosure.

In some embodiments, the subject has, or is at risk of having, gout, acute gout, acute intermittent gout, gouty arthritis, acute gouty arthritis, acute gouty arthropathy, acute polyarticular gout, recurrent gouty arthritis, chronic gout (with our without tophi), tophaceous gout, chronic tophaceous gout, chronic advanced gout (with our without tophi), chronic polyarticular gout (with or without tophi), chronic gouty arthropathy (with or without tophi), idiopathic gout, idiopathic chronic gout (with or without tophi), primary gout, chronic primary gout (with or without tophi), refractory gout, such as chronic refractory gout, axial gouty arthropathy, a gout attack, a gout flare, podagra (e.g., monarticular arthritis of the great toe), chiragra (e.g. monarticular arthritis of the hand), gonagra (e.g., monarticular arthritis of the knee), gouty bursitis, gouty spondylitis, gouty synovitis, gouty tenosynovitis, gout that affects tendons and ligaments, lead-induced gout (e.g., saturnine gout), drug induced gout, gout due to renal impairment, gout due to kidney disease, chronic gout due to renal impairment (with or without tophi), chronic gout due to kidney disease (with or without tophi), erosive bone disease associated with gout, stroke associated with gout, vascular plaque associated with gout, cirrhosis or steatohepatitis associated with gout, liver-associated gout, incident and recurrent gout, diabetes associated with damage to pancreas in gout, general inflammatory diseases exacerbated by gout, other secondary gout, or unspecified gout.

In some embodiments, the subject is suffering from acute gout, e.g., an acute gouty flare. Acute gouty flare is a serious debilitating condition causing excruciating pain with an unmet medical need. Despite several drugs on the market to manage chronic gout prophylactically, patients still suffer on average, one to three acute gout flare-ups every year that can last for 5-7 days.

Acute gouty flare can be an intensely painful and disabling inflammatory arthritis, that can involve a single joint or two or more joints. Without therapy, the gout flare may resolve completely within a few days to several weeks, particularly in early disease. However, symptoms can improve more quickly with administration of an anti-inflammatory drug. Upon resolution of a gout flare, the patient is said to have entered a symptom-free period. However, flares recur in the great majority of patients; with more frequent episodes, flares may be more severe and prolonged, with consequent shortening of asymptomatic periods.

Pain levels can be assessed by any method known in the art. In some embodiments, the pain level is assessed by clinical criteria, including self-reported pain scales, observation of patient behavior, and consideration of the impact of pain on daily life. Common tools include numeric rating scales (NRS), visual analog scales (VAS), and questionnaires like the Short Form McGill Pain Questionnaire (SF-MPQ). For example, a level of pain can be assessed in a human subject using a visual analog scale (VAS) score. A pain VAS score can be assessed by a subject indicating, e.g., on a scale from 0 (no pain) to 10 (worst pain ever experienced), pain before or after treatment with a composition of the present disclosure. In some embodiments, the pain VAS score is from 6 to 10, e.g., 6, 7, 8, 9, or 10, before treatment with a composition of the present disclosure. In some embodiments, the pain VAS score is from 0 to 5, e.g., 0, 1, 2, 3, 4, or 5, after treatment with a composition of the present disclosure. In some embodiments, the pain VAS score is reduced by from 1 to 10, e.g., 1, 2, 3, 4, 5, 6, 7, 8, 9, or 10, after treatment with a composition of the present disclosure compared with before treatment.

Inflammation levels can be assessed by any method known in the art. In some embodiments, the pain level is assessed by clinical criteria. For example, a level of inflammation can be assessed in a human subject using a visual analog scale (VAS) score. In a joint, e.g., an ankle joint suffering from an acute gout flare, inflammation can be assessed in comparison to a corresponding healthy ankle joint. An inflammation VAS score can be assessed by a subject indicating, e.g., on a scale from 0 (no difference from the other side) to 10 (very different from the other side). In some embodiments, the inflammation VAS score is reduced by from 1 to 10, e.g., 1, 2, 3, 4, 5, 6, 7, 8, 9, or 10, after treatment with a composition of the present disclosure compared with before treatment.

Other clinical treatment endpoints for inflammation or pain, including walking disability, sleep quality, mood changes, and treatment satisfaction can be assessed. In some embodiments, walking disability, sleep quality, mood changes, and/or treatment satisfaction can be assessed in a human subject using a visual analog scale (VAS) score.

In some embodiments, a level of walking disability can be assessed in a human subject using a visual analog scale (VAS) score. A walking disability VAS score can be assessed by a subject indicating, e.g., on a scale from 0 (no problem walking) to 10 (unable to walk). In some embodiments, the walking disability VAS score is reduced by from 1 to 10, e.g., 1, 2, 3, 4, 5, 6, 7, 8, 9, or 10, after treatment with a composition of the present disclosure compared with before treatment.

In some embodiments, a sleep quality level can be assessed in a human subject using a visual analog scale (VAS) score. A sleep quality VAS score can be assessed by a subject indicating, e.g., on a scale from 0 (normal sleep) to 10 (unable to sleep). In some embodiments, the sleep quality VAS score is reduced by from 1 to 10, e.g., 1, 2, 3, 4, 5, 6, 7, 8, 9, or 10, after treatment with a composition of the present disclosure compared with before treatment.

In some embodiments, a mood change level can be assessed in a human subject using a visual analog scale (VAS) score. A mood change VAS score can be assessed by a subject indicating, e.g., on a scale from 0 (normal mood) to 10 (feeling depressed, irritable or cannot concentrate). In some embodiments, the mood change VAS score is reduced by from 1 to 10, e.g., 1, 2, 3, 4, 5, 6, 7, 8, 9, or 10, after treatment with a composition of the present disclosure compared with before treatment.

In some embodiments, a treatment satisfaction level can be assessed in a human subject using a visual analog scale (VAS) score. A treatment satisfaction VAS score can be assessed by a subject indicating, e.g., on a scale from 0 (not likely) to 10 (definitely), how likely the subject would be to use the composition of the present disclosure instead of a known treatment. In some embodiments, the treatment satisfaction VAS score is from 1 to 10, e.g., 1, 2, 3, 4, 5, 6, 7, 8, 9, or 10, for a composition of the present disclosure.

In some embodiments, the method comprises treating pain. In some embodiments, the pain comprises post-operative pain.

The site of the surgery can have a profound effect upon the degree of post-operative pain a patient may suffer. In general, operations on the thorax and upper abdomen are more painful than operations on the lower abdomen, which in turn are more painful than peripheral operations on the limbs. However, any operation involving a body cavity, large joint surfaces, the spine or deep tissues should be regarded as painful. In particular, operations on the thorax or upper abdomen may produce widespread changes in pulmonary function, an increase in abdominal muscle tone and an associated decrease in diaphragmatic function. The result will be an inability to cough and clear secretions, which may lead to lung collapse and pneumonia. Prolonged pain can reduce physical activity and lead to venous stasis and an increased risk of deep vein thrombosis and consequently pulmonary embolism. In addition, there can be widespread effects on gut and urinary tract motility, which may lead in turn to post-operative ileus, nausea, vomiting and urinary retention. These problems are unpleasant for the patient and may prolong hospital stay. Many patients that experience moderate to severe post-operative pain, post-traumatic pain and burning pains, often require pain control at least in the first 3 days after trauma or surgery.

One known class of pharmaceuticals to treat post-operative pain is opioids. This class of compounds is well-recognized as being among the most effective type of drugs for controlling post-operative pain. Unfortunately, because opioids are administered systemically, the associated side effects raise significant concerns, including disabling the patient, depressing the respiratory system, constipation, and psychoactive effects such as sedation and euphoria, thereby instituting a hurdle to recovery and regained mobility. Further, because of these side-effects, physicians typically limit the administration of opioids to within the first 24 hours post-surgery. Thus, it would be preferable to use a non-narcotic drug such as ketorolac that delivers direct, localized pain control at a surgical site.

In some embodiments, the method comprises topically administering once, twice, or three times daily. In some embodiments, the method comprises topically administering three times daily.

As understood in the art, a dose is delivered each time the composition of the present disclosure is administered.

In some embodiments, topical administration comprises administering to the portion of the body of the subject suffering from inflammation or pain. In some embodiments, the topical administration comprises administering to the back. In some embodiments, the topical administration comprises administering to a joint.

A composition of the present disclosure may be administered to an individual in accordance with an effective dosing regimen for a desired period of time or duration, such as at least about 1 day, at least about 2 days, at least about 3 days, at least about 4 days, at least about 5 days, at least about 6 days, at least about a week, at least about 2 weeks, at least about 3 weeks, at least about one month, at least about 2 months, at least about 3 months, at least about 6 months, or at least about 12 months or longer. In some embodiments, the composition is administered for at least about 1 day, at least about 2 days, at least about 3 days, at least about 4 days, or for at least about 5 days. In some embodiments, the composition is administered on a daily or intermittent schedule for the duration of the subject's life.

Because the compositions of the present disclosure are believed to afford a safer toxicity profile, the methods and uses described herein can be used to treat patient populations that are contraindicated or are not recommended for use with approved ketorolac tromethamine methods, such as oral or intravenous. For example, Toradol is contraindicated in patients with serum creatinine concentrations indicating advanced renal impairment. Accordingly, in some embodiments, the subject is a patient having impaired renal function and/or a history of kidney disease.

Oral ketorolac tromethamine is not recommended for use in the last stages of pregnancy or during breastfeeding. The topical ketorolac compositions of the present disclosure are believed safe for use in these patient populations. In some embodiments, the subject is pregnant. In some embodiments, the subject is breastfeeding.

The dosage or dosing frequency of a composition of the present disclosure may be adjusted over the course of the treatment, based on the judgment of the administering physician.

The compounds and compositions of the present invention can be co-administered with other agents. Co-administration includes administering the compound or composition of the present invention within 0.5, 1, 2, 4, 6, 8, 10, 12, 16, 20, or 24 hours of the other agent. Co-administration also includes administering simultaneously, approximately simultaneously (e.g., within about 1, 5, 10, 15, 20, or 30 minutes of each other), or sequentially in any order. Moreover, the compounds and compositions of the present invention can each be administered once a day, or two, three, or more times per day so as to provide the preferred dosage level per day.

In some embodiments, co-administration can be accomplished by co-formulation, i.e., preparing a single pharmaceutical composition including the compounds and compositions of the present invention and any other agent. Alternatively, the various components can be formulated separately.

In some embodiments, a use of the present disclosure comprises a composition of the present disclosure for the manufacture of a medicament for aiding in the treatment of inflammation or pain in a subject in need thereof. In some embodiments, the use comprises a composition as described in Section IV above.

In some embodiments, a composition of the present disclosure is for use in aiding in the treatment of inflammation or pain in a subject in need thereof. In some embodiments, the compound for use comprises a pharmaceutical composition as described in Section IV above.

In some embodiments, a use of the present disclosure comprises a compound of the present disclosure or pharmaceutically acceptable salt thereof, or a pharmaceutical composition of the present disclosure for the manufacture of a medicament for treating inflammation or pain in a subject in need thereof. In some embodiments, the use comprises a composition as described in Section IV above.

In some embodiments, a compound of the present disclosure or pharmaceutically acceptable salt thereof, or a pharmaceutical composition of the present disclosure is for use in treating inflammation or pain in a subject in need thereof. In some embodiments, the compound for use comprises a composition as described in Section IV above.

Kits that comprise a composition of the present disclosure are also included in the present disclosure. In some embodiments, a kit further includes instructions for use. In some embodiments, a kit includes a composition of the disclosure, and a label and/or instructions for use of the composition in the treatment of the indications, such as the diseases or conditions, described herein. In some embodiments, kits comprising a composition of the present disclosure, or a pharmaceutically acceptable salt thereof, in combination with one or more (e.g., one, two, three, four, one or two, or one to three, or one to four) additional therapeutic agents are provided.

Provided herein are also articles of manufacture that include a composition of the present disclosure or a pharmaceutically acceptable salt thereof in a suitable container. In some embodiments, the container is a vial, jar, tube, or ampoule.

### VI. Examples

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

### Example 1. Calculations of Ketorolac Penetration

Key transdermal indicators were computed using industrial molecular modelling software (from Biovia) and comparing the results obtained for the two drugs ibuprofen and ketorolac.

The Biovia software integrates quantum chemistry, dielectric continuum models, electrostatic surface interactions (computed with DFT-Turbomole) and statistical thermodynamics. This combined approach was used to study the penetration of APIs through the Skin Stratum Corneum.

Molecular weight and the computed Log P values for the drugs are shown below:

| | Mw | Log P (of Neutral API forms) |
|---|---|---|
| Sodium Ibuprofen (SIB) | 228.26 | 3.84 |
| Ketorolac Tromethamine (KT) | 376.4 | 3 |

The molecular weight (Mw) of KT is ~40% larger than that of SIB. Even when looking at the neutral forms ketorolac and ibuprofen, ketorolac is more hydrophilic than ibuprofen as shown by the computed Log P values above. Ketorolac is less likely to penetrate the skin.

To better understand the transdermal composition requirements, computer simulations were carried out using the Biovia software for the following transdermal markers:
1) Active Pharmaceutical Ingredient (API) Free Energy Profile within the Long Periodicity Phase (LPP) motif of the Stratum Corneum (SC) modelled for Human Skin. The LPP motif is the primary SC motif barrier and it was expected to be the limiting factor to Transdermal Delivery through SC. To obtain the API free energy the API molecule is pulled through the LPP motif in a computer simulation. The API molecule is equilibrated in different sub-layers of the LPP motif and the space dependent API Free Energy is calculated in each sub-layer. The calculation is carried out for half the LPP motif only since that motif is symmetric down the middle.
2) API Spatial Distribution through the LPP motif in SC. This indicator tells us the relative probability of finding the API in the LPP motif and points to where the potential barriers for the TD are located in the LPP motif.
3) API Entropy within the Long Periodicity Phase. That indicates the locations where the API will spend the larger amount of time (regions with the largest amount of API conformations) in the LPP motif.
4) Resistance Indicates the resistance of the LPP motif to API penetration and it is given by the Log of the inverse of the API steady state flux through the LPP motif normalized by concentration.

The Free Energy (FE) Profiles of the API through half LPP motif were computed using a molecular model for the LPP SC motif. The calculation included the electrostatic surface charges of all molecules in the LPP (Ceramides, Cholesterol and Free Fatty Acids) and their interactions with the API. Results for the FE of Ibuprofen and Ketorolac are given below. The converged morphology of the LPP SC motif without API matches well the experimental X ray data and the expected LPP size (FIG. 1).

To obtain the API Free Energy the API molecule was first placed on the right side of the LPP motif (x=60A) in a water layer and was pulled through the LPP motif until the middle of the LPP motif was reached (at x=0A). To single out the effect of the LPP on the APIs the FE Reference State for each API in the water phase was taken to be 0 by default.

The left side of the graph x axis corresponds to the middle of the LPP motif. Note both APIs faced two barriers to LPP penetration but the ketorolac barrier was higher by around 1.5 kcal/mol (which is larger than twice RT at room temperature-R is the gas constant). It is likely different formulation approaches to ketorolac and ibuprofen are required.

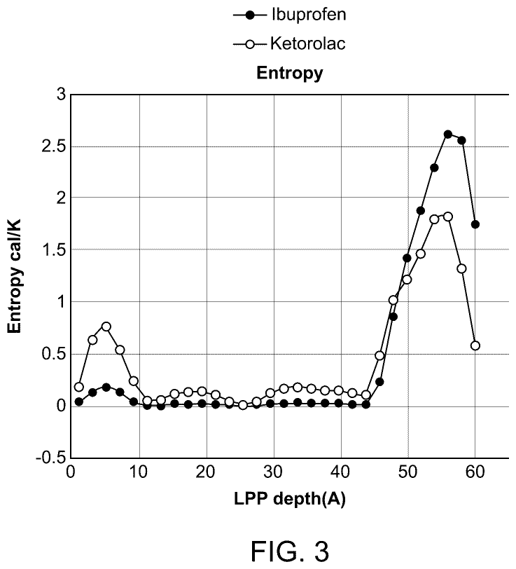

FIG. 2 shows API distribution in LPP motif in stratum corneum. Here, ketorolac had a harder time than ibuprofen in crossing the LPP surface layer and it had as a smaller presence in the middle of the half LPP motif (x=~25A). Ketorolac had a harder time moving through the LPP motif than ibuprofen. Ketorolac had a higher affinity for the entropic hole present in mid LPP motif (at x=0A).

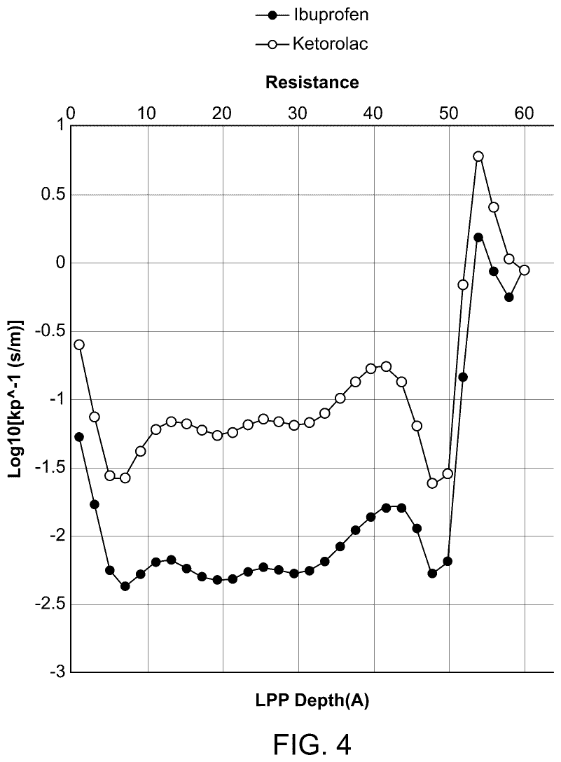

To further substantiate these effects, the computed API entropy was determined (FIG. 3). Ibuprofen had a larger entropy while passing through the LPP motif surface while ketorolac was more likely to get stuck in the middle of the LPP due to the entropy hole (larger entropy for ketorolac).

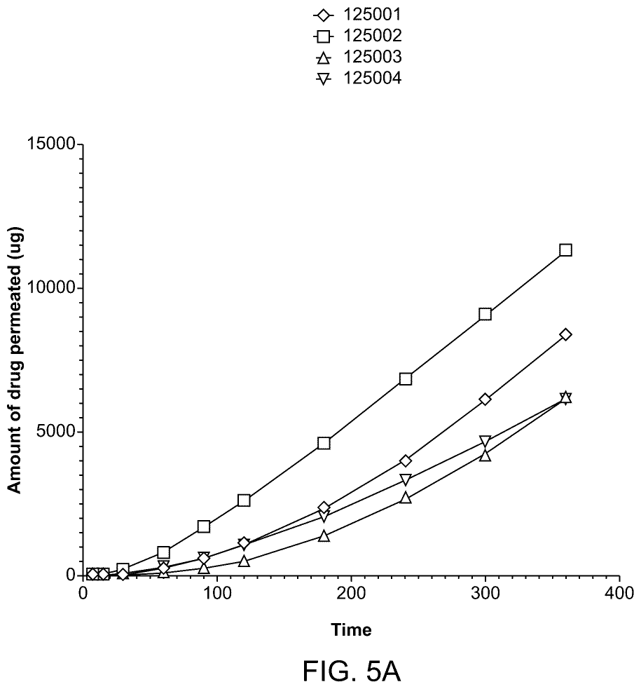

LPP resistance difference was compared to API penetration (FIG. 4). Note this transdermal marker included both kinetic and thermodynamic contributions to transdermal penetration. The permeability coefficient kp=the steady-state flux normalized by the concentration. Note ibuprofen due to its smaller size, less rigidity and less charges faces 91% less resistance in skin penetration in the majority region of the LPP motif. A factor of ~100 reduction of the density normalized steady state flux through the LPP motif of the SC between ketorolac to ibuprofen was calculated. While ibuprofen and ketorolac are in the same NSAID class they are likely in different transdermal formulation development classes and their transdermal optimized composition will require a different approach.

### Example 2. Ketorolac Composition Preparations

The following compositions were formulated according to the methods described herein. Generally, the eutectic solvent was prepared first by heating choline chloride and propylene glycol, followed by addition of the remaining ingredients.

### Preparation of Composition 125010

Propylene glycol (1575 kg) was placed in a 5 L glass beaker. To this was added choline chloride (781.01 kg) with stirring at 55-75° C., with target temperature of 65° C., until all particulates dissolved. The premixed eutectic solvent mixture was maintained at about 70° C. until the ketorolac mixture was prepared as follows.

Glyceryl monostearate (280 kg) was placed in a 10-L glass beaker with overhead stirring at 55-70° C. To the beaker was added SPAN 80 (sorbitan oleate, 8 kg) and polysorbate 80 (72 kg) while the temperature was maintained at 55-70° C. Dimethyl sulfoxide (784 kg) was added to this mixture with stirring, while the temperature was maintained at 65-75° C. Ketorolac tromethamine (500 kg) was slowly added to the mixture with stirring. The resulting ketorolac mixture was warmed to 68-71° C., targeting 70° C.

The premixed eutectic solvent mixture at 70° C. was then added to the ketorolac mixture at 70° C. with overhead stirring at about 100-300 rpm. Once combined, the resulting Composition 125010 was allowed to cool to room temperature with stirring at low speed overnight.

Other compositions shown in Table 1, Table 2A, and Table 2B were prepared in a similar manner as described for Composition 125010.

TABLE 1
Ketorolac compositions shown as percentage composition by weight

| | 125001 | 25001 | 25002 | 25003 | 25004 | 25010* | 25011^{§} | 25012 | “B” | “C” |
|---|---|---|---|---|---|---|---|---|---|---|
| ketorolac | 12.5 | 2.5 | 2.5 | 2.5 | 2.5 | 2.5 | 2.5 | 2.5 | 3.75 | 7.5 |
| tromethamine | | | | | | | | | | |
| choline | 22.5 | 33.5 | 26 | 36.8 | 43.4 | 6.062 | — | 20.8 | 36.8 | 35.4 |
| chloride | | | | | | | | | | |
| propylene | 45.4 | 36.6 | 52 | 30.1 | 23.5 | 26.975 | — | 41.6 | 40.2 | 38.6 |
| glycol | | | | | | | | | | |
| propylene | — | — | — | — | — | 12.932 | — | — | — | — |
| glycol | | | | | | | | | | |
| slurry | | | | | | | | | | |
| dimethyl | 19.6 | 17.6 | 19.6 | 17.6 | 17.6 | — | — | 15.6 | 19.3 | 18.5 |
| sulfoxide | | | | | | | | | | |
| Laureth- | — | 10 | — | 10 | 10 | — | 20.5 | 19.5 | — | — |
| 23 | | | | | | | | | | |

*Composition 25010 also included 3.917% glyceryl monosterate, 3.917% cetyl alcohol, 2.549% oleic acid, 2.549% polysorbate 20, 3.917% squalene, 1.293% dimethacone, 1.293% xanthan gum, and 32.094% water.
^{§}Composition 25011 also included 77.5% water.

TABLE 2A
Ketorolac compositions shown as percentage composition by weight

| | 125002 | 125003 | 125004 | 125010^{§} | 25013^{¥} |
|---|---|---|---|---|---|
| ketorolac | 12.5 | 12.5 | 12.5 | 12.5 | 2.5 |
| tromethamine | | | | | |
| choline | 20.9 | 25.7 | 24.0 | 19.5 | 13.448 |
| chloride | | | | | |
| propylene | 42.1 | 51.8 | 48.5 | 39.4 | 14.658 |
| glycol | | | | | |
| dimethyl | 19.6 | 10 | 10 | 19.6 | — |
| sulfoxide | | | | | |
| Laureth-23 | 5 | 0 | 5 | 0 | 5.621 |

^{§}Composition 125010 also included 7.0% glyceryl monostearate, 0.2% SPAN 80 (surfactant for homogenizing gel) and 1.8% polysorbate 80 (surfactant for homogenizing gel).
^{¥}Composition 25013 also included 5.621% glyceryl monosterate, 5.621% cetyl alcohol, 3.373% oleic acid, 5.621% polysorbate 20, 5.621% squalene, 6.745% dimethacone, 0.787% xanthan gum, 3.373% isopropyl myristate, and 28.106% water.

TABLE 2B
Ketorolac compositions shown as
percentage composition by weight

| | 125010 | 125011 | 125012 | 125013 | 125016 | 125018 |
|---|---|---|---|---|---|---|
| ketorolac | 12.5 | 12.5 | 12.5 | 12.5 | 12.5 | 12.5 |
| tromethamine | | | | | | |
| choline | 19.5 | 0 | 22.5 | 29.0 | 38.1 | 9.1 |
| chloride | | | | | | |
| propylene | 39.4 | 0 | 45.4 | 58.5 | 20.8 | 49.8 |
| glycol | | | | | | |
| dimethyl | 19.6 | 78.5 | 19.6 | 0 | 19.6 | 19.6 |
| sulfoxide | | | | | | |
| glyceryl | 7 | 7 | 0 | 0 | 7 | 7 |
| stearate | | | | | | |
| sorbitan oleate | 0.18 | 0.18 | 0 | 0 | 0.18 | 0.18 |
| (SPAN 80) | | | | | | |
| polysorbate 80 | 1.8 | 1.8 | 0 | 0 | 1.8 | 1.8 |

### Example 3. In Vitro Studies on Ketorolac Composition Penetration

The following compositions were tested for in vitro penetration in Logan Franz cell protocol: 125001, 125002, 125003, 125004, 125010, 125011, 125012, 125013, 125016, and 125018 from Example 2. The Logan Instrument Automated Franz Cell was utilized for this experiment with receptor fluid containing 91 (50 mM tris HCl 0.15M NaCl): 9 (Isopropanol) ratio. Strat M Membranes were then fitted to the receptor compartments and secured with a dosing disk pressed into a donor compartment and a clamp. The cells were then all filled with 12 mL of receptor fluid and 1000 μL of solution was added to the donor compartment using a micropipette. The Logan Instrument then took 1 mL samples at 7, 15, 30, 60, 90, 120, 180, 240, 300, and 360 minutes.

HPLC Instrument Protocol: The HPLC was dry primed, wet primed, and the injector was purged before equilibrating and heating the column to 45° C. using the ketorolac Iso Method. This method uses a degassed buffer containing 55:45 50 mM Ammonium dihydrogen phosphate buffer at pH 3.01: Methanol at a flow rate of 1 mL/min. Each sample runs for 8.5 minutes with the Ketorolac peak occurring at about 7.4 minutes. Injection volume: 10 μL. A standard curve was established and the concentration of samples was calculated using the equation established by the standard. Each sample from the Logan machine was run using the HPLC protocol and a concentration vs time curve was generated. Results were summarized in FIG. 5A-5C.

DMSO was observed to increase absorption through the membrane. Composition 125001 had proportionately double the concentration of DMSO as Composition 125003. Similarly 125002 had proportionately double the concentration of DMSO as Composition 125004. In both cases, compositions with increased concentrations of DMSO yielded significantly higher absorption rates. See, FIG. 5A.

The addition of glyceryl monostearate (GMS), in the form of Laureth-23, increased the absorption rate through the membrane. Composition 125002 had an addition of GMS in the composition compared to 125001. Similarly 125004 had an addition of GMS in the composition compared to 125003. In both cases, compositions with GMS yielded higher absorption rates.

Compositions 125010, 125012, and 125013 containing a eutectic solvent comprising choline chloride and propylene glycol exhibited significantly higher ketorolac permeability through the membrane compared with Composition 125011 that contained DMSO but lacked the eutectic solvent. The greatest effect on ketorolac delivery was observed between the composition having ketorolac and DMSO only, that is, without a eutectic solvent, and those having ketorolac and the eutectic solvent choline chloride and propylene glycol. The presence of glyceryl stearate and surfactants did increase ketorolac permeability (c.f. 125010 to 125012) as did the presence of DMSO (c.f. 125010 to 125013). Each of Compositions 125010, 125012, and 125013 delivered 1 mg or more ketorolac across the membrane after 4 hours, as compared to Composition 125011, which failed to deliver greater than 1 mg ketorolac even after 8 hours. See, FIG. 5B.

A specific ratio of the eutectic components choline chloride and propylene glycol was not required to deliver ketorolac across membranes. A range of molar ratios of the components choline chloride and propylene glycol in Compositions 125010 (1:3.7 choline chloride: propylene glycol), 125016 (1:1 choline chloride: propylene glycol), and 125018 (1:10 choline chloride: propylene glycol) afforded mixtures that delivered greater than 1 mg ketorolac across the membrane over 4-8 hours. See, FIG. 5C.

### Example 4. Comparative In Vitro Studies on Ketorolac Composition Penetration

The following compositions were tested for in vitro penetration in Logan Franz cell protocol: Composition 125010 from Example 2 as well as comparative ketorolac compositions containing an ionic liquid (I1) and betaine/lactic acid eutectic (BLA1).

| | Magnesium chloride | Betaine/lactic acid |
|---|---|---|
| | ionic liquid (I1) | composition (BLA1) |
| Composition | (w/w %) | (w/w %) |
| ketorolac tromethamine | 12.5 | 12.5 |
| DMSO | 19.6 | 19.6 |
| GMS | 7.0 | 7.0 |
| SPAN80 | 0.18 | 0.18 |
| TWEEN80 | 1.82 | 1.82 |
| Magnesium chloride | 53.4 | — |
| hexahydrate | | |
| Water (deionized) | 5.5 | — |
| Betaine | — | 30.6 |
| Lactic acid (88.6%) | — | 28.3 |

In vitro study design: All formulations contained the same amounts of ketorolac, dimethyl sulfoxide (DMSO), glyceryl monostearate (GMS), SPAN80®, and TWEEN80®. The ionic liquid and eutectic solvent components across the three formulations had equal weight percents. Ketorolac tromethamine was soluble and compatible with the component combinations of all three formulations.

Logan Franz Cell Protocol: an Automated Transdermal Diffusion Sampling Cell System (dry heat; 12-cells) by Logan Instruments was utilized for this experiment with receptor fluid containing isotonic phosphate buffered saline. Strat M Membranes, sealed with PTFE and double-sided tape gaskets with internal diameter hole of 1.5 cm, were then fitted to the receptor compartments and secured with a dosing gasket pressed into a donor compartment and a clamp. The cells were then all filled with 12 mL of receptor fluid and 1.0 g of test gel was added to the donor compartment using a syringe. The Logan Instrument then took 1 mL samples at 0.12, 1, 2, 3, 4, 5, 6, 7 and 8 hours.

HPLC Instrument Protocol: The HPLC was dry primed, wet primed, and the injector was purged before equilibrating and heating the column to 45° C. using the Ketorolac Isocratic Method as follows. This method used a C18 column (150 cm×4.6 mm×5 μm) with a degassed buffer containing 55:45 50 mM Ammonium dihydrogen phosphate buffer at pH 3.01: Methanol at a flow rate of 1 mL/min. Each sample was evaluated for 8.5 minutes with the Ketorolac peak occurring at about 6.5 minutes at detection wavelength 323 nm. Injection volume was 10 μL.

A standard curve was generated by preparing 6-8 ketorolac tromethamine reference standard solutions of differing concentrations over the entire assay range and analyzed by using the HPLC protocol. Each sample from the Logan machine is analyzed by using the HPLC protocol and then the ketorolac concentration in each sample is calculated by using the established absorbance vs concentration curve. From the sample ketorolac concentration data, a concentration vs time curve is generated for each test article sample. Each sample was tested in four Franz cells of the Logan instrument.

The data from this experiment demonstrated that the rate and cumulative ketorolac transfer of 125010 was higher than a composition comprising a less compatible eutectic (BLA1) for ketorolac transdermal permeability (FIG. 6). Additionally, this experiment demonstrated that the rate and cumulative ketorolac transfer of 125010 was higher than a formulation consisting of an ionic liquid (IL). This data provided experimental evidence demonstrating the effect of differences in chemical, physical and thermodynamic properties between eutectic mixtures, as well as a strong ionic liquid compared with a eutectic mixture that has been designed for ketorolac.

### Example 5. Rat Pharmacokinetics of Topically Administered Ketorolac Composition

Absolute Plasma Bioavailability Study Design: Thirty six rat subjects were assigned evenly to six groups; groups 1-5 which were single dose application of 25002, 25011, 25012, 25013, and group 6 which was a multiple dose application of 25012 (see Tables 1, 2A, and 2B). 2 g of test article was applied to the back of each rat. A series of blood samples were collected from each group at 0 hr, 1 hr, 2 hr, 4 hr, 8 hr, 24 hr and 36 hr to quantify the ketorolac plasma concentration profile over time.

A composition containing no eutectic solvent delivered on average significantly less ketorolac to rat plasma when compared to compositions containing a eutectic solvent (FIG. 7). All compositions in this experiment contained the same percent of ketorolac tromethamine of 2.5%. Composition 25011 (shown in red in the graph below) delivered markedly less ketorolac when compared with 25002, 25012, and 25013, all of which contained a eutectic solvent.

### Example 6. In Vivo Rat Study on Ketorolac Composition Effect on Pain Sensitivity

Paw Pressure Study Design: After acclimation and a pre-injury baseline test for Paw Withdrawal threshold (PWT), rats were anesthetized and 50 μL of Freund's Complete Adjuvant (FCA) mixture (25 μL of FCA diluted 1:1 with saline) was injected into the left hind paw. Approximately 24 hours after FCA injection, baseline pre-dose paw withdrawal thresholds were assessed using a paw pressure device. The behavioral endpoint was defined as paw withdrawal. These baseline thresholds were used to assign rats to treatment groups in a stratified, random fashion as much as possible.

Test compositions included “B” and “C” of Table 1 above. A control composition “A” included choline chloride (38.27%), propylene glycol (41.72%), and dimethyl sulfoxide (20%), with no ketorolac tromethamine.

Test substances were administered by dermal application. The back of the animals were shaved over an area of the lumbar region lateral to the L4 and L5 lumbar vertebral bodies, extending at least 0.5 inches to either side of the midline Each rat was tested for Paw Withdrawal Threshold (PWT) 15, 30, 60, 120, 180 and 240 minutes after dose administration.

To measure the PWT, this study used the UGO Basile Analgesy device which measured the force on a rat or mouse paw according to the Randall-Selitto paw-pressure test. The test increased mechanical pressure applied to the surface of the paw until withdrawal or vocalization occurred. This test measured nociceptive thresholds. In this model, high PWT values were indicative of decreased pain and low PWT values are indicative of increased pain.

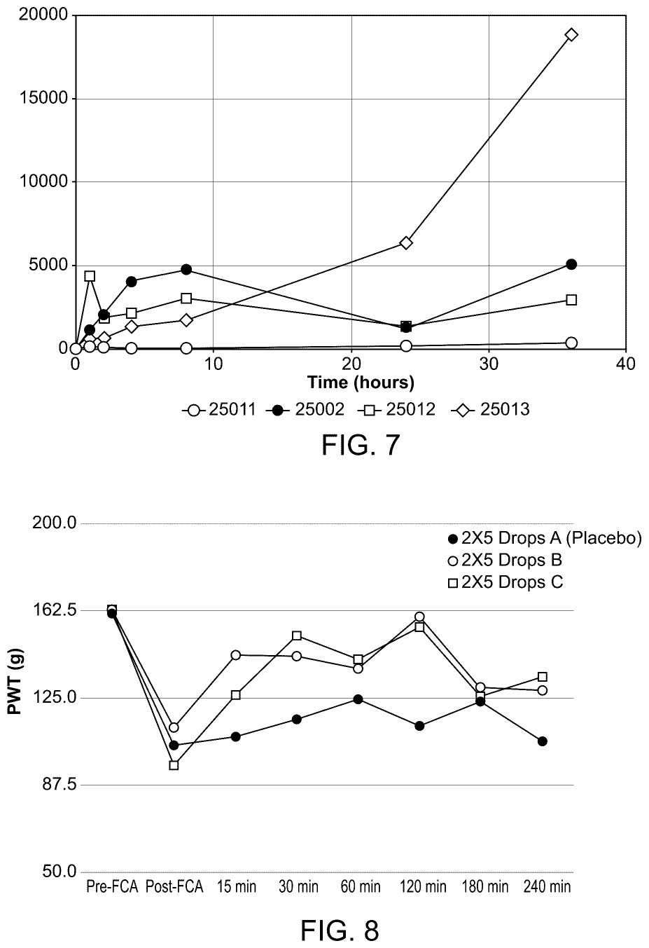

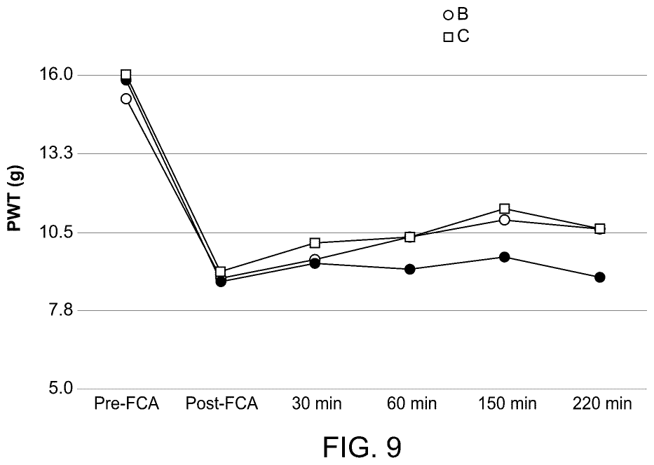

Topical compositions “B” or “C” reduced pain sensitivity when compared to negative control (FIGS. 8 and 9). At 120 minutes, compositions “B” or “C” achieved comparable pain tolerance to pre-FCA administration baseline.

### Example 7. Single Dose In Vivo Pig Study on Ketorolac Composition Bioavailability

Composition 25002 was tested for absolute bioavailability in plasma and synovial fluid in a pig model. Pig was considered a good transcutaneous model, as being most comparable to human skin permeability and joint tissue and structure. Induction of joint inflammation has been well-characterized in rodent and dog models. This study investigated this model in young Yorkshire swine. Test article was administered at peak inflammation 3-3.5 hours after induction. For ethical and data integrity reasons, the swine were anesthetized during inflammation induction, treatment and sample collection procedures.

Absolute Plasma Bioavailability Study Design: Eight porcine subjects were assigned evenly to two groups; control (1 mg/kg single intravenous bolus administration, Group 01) or test (10 g single transcutaneous administration, Group 02) article 25002. 5 g of test article was applied to each hind hock. A series of blood samples were collected from each group within the first 24 hours to quantify the ketorolac plasma concentration profile over time. No inflammation induction nor synovial fluid was collected during this portion of the study.

Absolute Bioavailability in Synovial Fluid Study Design: After a 7 day wash out period, control and test article were administered to animals where inflammation was induced in one hind hock at peak of inflammatory response. Peak inflammatory response was observed after 3-4 hours and confirmed prior to dose administration. Synovial fluid samples were collected from each hind hock at 60, 120 and 180 minutes, with matching whole blood samples. Whole blood sample just prior to dose administration was also collected.

Reactive Protein-C(CRP) Inflammatory Response Study: Whole blood samples were collected for the purposes of measuring C-reactive protein response as a surrogate biomarker of inflammation efficacy.

Composition 25002 was found to penetrate deeper tissues to deliver significantly higher local ketorolac concentrations than systemic circulation (FIG. 10). From 60 to 180 minutes after topical administration, synovial concentrations of ketorolac were higher than in plasma.

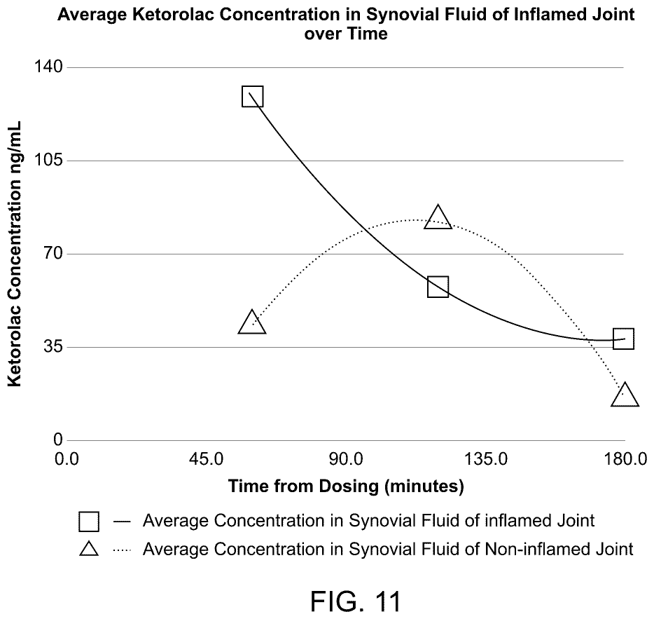

Composition 25002 delivered higher ketorolac concentrations to inflamed joints than noninflamed joints. (FIG. 11).

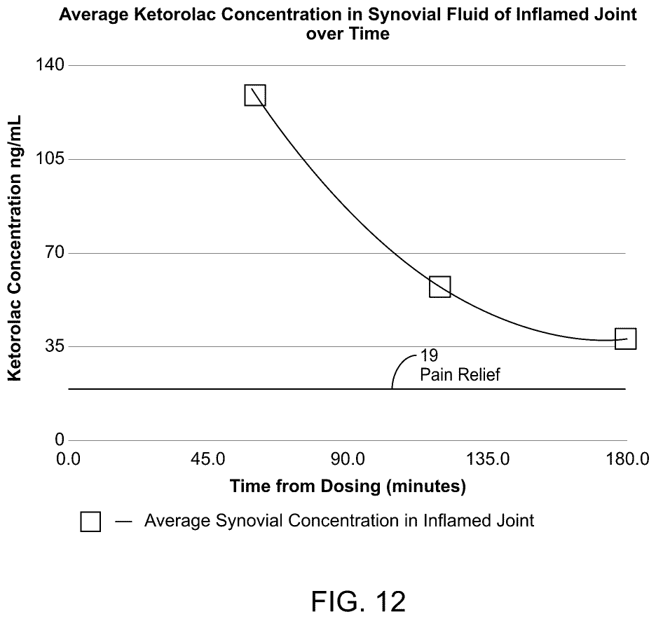

Composition 25002 delivered local pain relief within 60 minutes of administration and was maintained for at least 3 hours following dosing on average (FIG. 12). The calculated efficacy, as identified by ketorolac IC₅₀ for the cyclo-oxygenase enzyme, is 19 ng/ml for pain relief (Warner, T. D. et al. Proceedings of the National Academy of Sciences 1999, 96 (13), 7563-7568). The average synovial fluid ketorolac concentration was sustained above this level for all sample collection time points; 129 ng/mL (60 minutes), 57 ng/ml (120 minutes) and 38 ng/ml (180 minutes).

### Example 8. Multiple Dose In Vivo Pig Study on Ketorolac Composition Bioavailability

Composition 125010 was tested to quantify the effect of multiple daily doses of 125010 on penetration to deeper tissues by measuring synovial fluid ketorolac concentrations in inflamed joints.

Study Design: Seven porcine subjects were assigned to three groups. Group 1 was a control group of one animal administered an intravenous 1 mg/kg bolus dose, identical to the Single Dose Porcine POC Study. The remainder of the animals were evenly assigned to Groups 2 and 3, which studied two dose volumes of 125010 dosed three times in 4-hour intervals. There were 3 synovial fluid sample collections after each topical dose administration, with matching whole blood samples. Baseline whole blood samples were collected prior to first dose administration, and at the additional time points listed in the Table of Samples, below, in order to accurately quantify the topical plasma PK profile for this three times daily (TID) dose regimen.

| Group | Treatment |
|---|---|
| 1 | Control Article |
| | (1 mg/kg QD dose of 30 mg/mL IV ketorolac |
| | tromethamine solution) |
| 2 | 10 g per hind hock, one dose every 4 hours |
| | three times of 125010 |
| 3 | 5 g per hind hock, one dose every 4 hours |
| | three times of 125010 |

This study focused on ketorolac delivery to inflamed joints. Inflammation was induced in both hind hock joints of each animal.

Plasma and synovial fluid samples were taken periodically. Time zero was defined as the time of inflammation induction and time 2 hours was the time of initial dose administration. The 1 mg/kg IV bolus dose was administered only once as a synovial fluid and plasma ketorolac concentration control measurement.

Composition 125010 delivered local analgesia, or total elimination of pain, within 60 minutes of administration and pain relief was maintained for at least 11 hours. Pain relief has been associated in literature with ketorolac IC₅₀ of the cyclo-oxygenase (COX) enzyme at 19 ng/mL. Freedom from pain, or analgesia, is associated with ketorolac IC₈₀ of COX enzyme at 244 ng/ml. See, Hunter, T. S., et al. American Journal of Managed Care 2015, 21 (7), S139-S147.

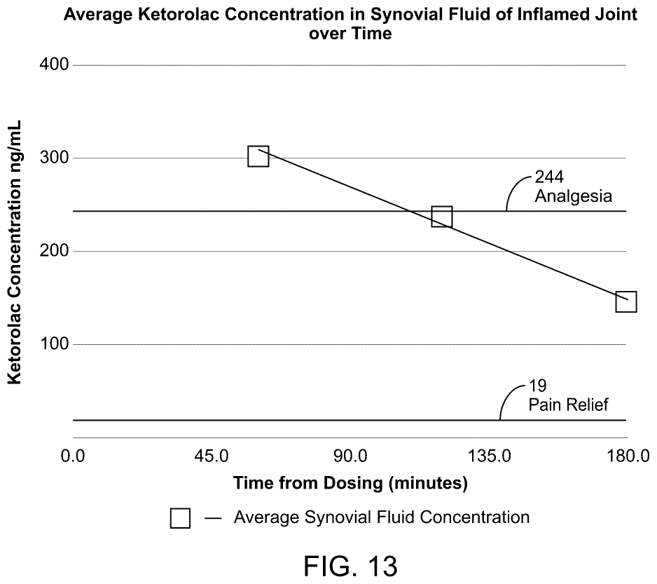

The earliest time point information available was 60 minutes post-dose administration of 125010 from the Porcine Single Dose POC Study according to the protocol of Example 6. The average concentration in synovial fluid for both inflamed and non-inflamed joints in the porcine single dose POC study was 303 ng/ml (60 minutes), 239 ng/mL (120 minutes) and 146 ng/mL ((180 minutes) (FIG. 13).

The synovial fluid ketorolac concentration data from this study supports this finding where the average Group 3 synovial fluid ketorolac concentration for all joints was 328 ng/ml (120 minutes) and 239 ng/ml (180 minutes) (FIG. 14). Ketorolac synovial fluid delivery was comparable or better for 125010 than 25002. The duration of effect was supported up to 11 hours for three 125010 doses delivered at 4-hours intervals by this study, consistent with freedom from pain or near-freedom from pain.

Composition 125010 delivered local pain relief, based on ketorolac COX-1 IC₅₀ and COX-2 IC₈₀, while minimizing plasma concentrations to avoid the well-characterized ketorolac-induced adverse effects associated with higher ketorolac systemic exposure (FIG. 15). The average maximum ketorolac plasma concentration for three transcutaneous doses was 18-fold lower than the maximum ketorolac plasma concentration of a single IV bolus dose of 1 mg/kg, and AUC (0-24 h) was ~1.4-fold lower. Local average synovial fluid ketorolac concentrations were comparable to a single IV bolus dose.

### Example 9. Clinical Study on Topical Ketorolac Composition Effect on Acute Gouty Flare

Composition 125010 was tested in a phase 1 safety, tolerability, and pharmacokinetic study compared to intravenous ketorolac tromethamine in adult healthy volunteers and gout patients.

Acute gouty flare can be an intensely painful and disabling inflammatory arthritis, usually involving a single joint but occasionally involving two or more joints. Without therapy, the gout flare may resolve completely within a few days to several weeks, particularly in early disease. However, symptoms can improve more quickly with administration of an anti-inflammatory drug. Upon resolution of a gout flare, the patient is said to have entered a symptom-free period. However, flares recur in the great majority of patients; with more frequent episodes, flares may be more severe and prolonged, with consequent shortening of asymptomatic periods.

Systemic non-steroidal anti-inflammatory drugs (NSAIDs) are a primary treatment choice for acute flare of gout. The goal of therapy in a gout flare is prompt and safe termination of pain and disability. But systemic NSAIDs have significant issues and are generally contraindicated in the older gout population, especially those individuals with co-morbidities such as gastrointestinal (GI) problems, bleeding disorders and reduced kidney function. In summary, these current treatments for acute gouty flare can become largely ineffective or unsuitable and are associated with significant adverse events.

Local, topical administration of 125010 composition (transcutaneous ketorolac tromethamine gel, 12.5% (w/w)) may provide a viable alternative by lowering systemic absorption, avoiding passage through the GI system and still providing pain relief associated with NSAIDs at the affected joints. By targeting treatment to only the area of flare-up, the inflammation and pain can both be treated, while potentially reducing the systemic side effects and provide an option to those with GI issues.

### Objectives

The objectives of this study were to evaluate the safety, tolerability, and bioavailability of topically applied 125010 in adult healthy volunteers, compared to intravenous administration of ketorolac tromethamine injection, USP.

Once safety and tolerability in adult healthy volunteers has been assessed, this study also evaluated the safety and tolerability of 125010 in adult gout participants experiencing a gout flare.

Data relating to symptomatic relief of gout flare up was collected for the purposes of proof-of-concept study design.

### Study Design

This study was a phase 1, open-label, bioavailability, safety and PK study of topically applied transcutaneous ketorolac tromethamine gel 12.5% (w/w) (125010) versus intravenous administration of approved ketorolac tromethamine injection, USP (15 mg/mL) comparator in healthy volunteers, including an evaluation of safety, tolerability, and efficacy in gout participants with flare-up. Participants were in the age range of 18-64 years of age (Cohort 1) and 18-70 years of age (Cohorts 2 and 3). This study was conducted in three consecutive cohorts. Cohorts 1 and 2 were conducted inpatient, while Cohort 3 was conducted outpatient.

Cohort 1: Open-label, single-day, multiple-dose (three doses at 0.6 g, 2.1 g, and 5.0 g per dose), intra-subject bioavailability, PK, safety and toleration, dose-escalation design in approximately 8 adult healthy volunteers. Study participants were admitted to the clinic and received three doses of topically administered drug product over the course of 1 day, 6 hours apart for each of the 4 treatment periods of this cohort, except the first treatment period in which one dose of intravenously administered drug product was administered. Escalation to the next dose proceeded unless stopping criteria were met. The next dose began after a 2-day wash-out period, and there were three dose levels for the topically applied transcutaneous ketorolac tromethamine gel 12.5% (w/w) (125010) compared to 30 mg dose of intravenous administration of ketorolac tromethamine injection, USP (15 mg/mL). Total in-clinic time was 12 days, with discharge occurring the morning of Day 12. Note that study participants were admitted to the clinic on Day −1. Thus, study participants were admitted during 13 calendar days, and stayed overnight for 12 nights.

Safety Review Committee meeting reviewed all available Cohort 1 safety and ketorolac PK data and approved the dose regimen for Cohort 2.

Cohort 2: Open-label, 1-arm, 5-day, multiple-dose (three doses at 0.6 g, 2.1 g, and 5.0 g per dose) PK, safety, and toleration in approximately 12 adult healthy volunteers at doses not to exceed the maximum tolerated dose demonstrated by Cohort 1. Study participants were admitted to the clinic and received three doses of drug product, 6 hours apart, over the course of 5 days. There were two dose levels for the topically applied transcutaneous ketorolac tromethamine gel 12.5% (w/w) (125010). Cohort 1 participants were eligible to participate in Cohort 2. Total in-clinic time was 7 days.

Safety Review Committee meeting reviewed all available Cohort 1 and 2 safety and ketorolac PK data and approved continuance to Cohort 3.

Cohort 3 was an open-label, 1-arm, 5-day, multiple-dose (three doses at 5.0 g per dose) safety, toleration, and efficacy design in ten adult gout participants with flare-up. For Cohort 3, the initial dose was administered as early in the morning as practical, so that the study participant had the opportunity to apply the study medication three times in study day 1. The study participant applied the second dose and beyond. The study participant dosing instructions required doses to be applied in the morning, then at +6 hours (+1 hour) and +12 hours (+1 hour) of the morning dose for each of the study days.

### Study Treatments

Composition 125010 and ketorolac tromethamine injection, USP (15 mg/mL) were provided to the clinical site.

Cohort 1: Composition 125010 was provided as bulk topical gel, each dose weighed out by the clinic pharmacist prior to dose administration for each study participant by qualified site personnel. Ketorolac tromethamine injection, USP (15 mg/mL) was provided as an over-labeled US-based commercial supply. Dose was calculated and measured by a clinical pharmacist prior to dose administration for each study participant by qualified site personnel.

TABLE 3
Cohort 1 Schedule Overview by Treatment Period

| Period 1 (30 mg | | | Period 2 (0.6 g | | | Period 3 (2.1 g | | | Period 4 (5.0 g | | |
|---|---|---|---|---|---|---|---|---|---|---|---|
| IV bolus, QD) | | | topical, TID) | | | topical, TID) | | | topical, TID) | | |
| D 1* | D 2 | D 3 | D 4 | D 5 | D 6 | D 7 | D 8 | D 9 | D 10 | D 11 | D 12 |

| | | | | | | | |
|---|---|---|---|---|---|---|---|
| dose | washout | dose | washout, SRC | dose | washout, SRC | dose | washout, |

| clinical data | clinical data | discharge |
|---|---|---|
| review/decision | review/decision | |

*Day of treatment, e.g., D1 = Day 1, D2 = Day 2, etc.; SRC = safety review committee.

Cohort 2: Composition 125010 was provided as bulk topical gel, each dose weighed out by the clinic pharmacist prior to dose administration for each study participant by qualified site personnel.

Cohort 3: Composition 125010 was provided as individual dosing containers with dose applicator dispensed by the clinic pharmacist at the beginning of the study to each study participant for dose administration by the study participant. The first dose was under the supervision of qualified site personnel.

### Study Eligibility

This section provides an overview of the main inclusion criteria.

Cohorts 1 and 2: For Cohort 1, study participant age was 18 to 64 years of age, inclusive, at the time of consent. For Cohort 2, study participant age was 18 to 70 years of age, inclusive, at the time of consent.

Body mass index (BMI) from 18.0 to 32.0 kg/m², inclusive; and body weight of at least 50 kg (110 lbs). [BMI=weight (kg)/(height (m)²)]. Healthy as determined at screening by the investigator based on medical history, physical examination, clinical laboratory tests, 12-lead electrocardiogram measurements and vital sign measurements. Resting supine systolic blood pressure from 90 to 140 mm Hg at screening, inclusive and resting supine diastolic blood pressure from 40 to 90 mm Hg, inclusive.

Male participants agreed to utilize a barrier contraceptive method for the duration of the study and agreed to avoid sperm donation for at least 90 days after completing study participation.

Female participants were of non-childbearing potential (defined as either surgically sterile or at least 1 year post-menopausal; post-menopausal was defined as no menses for 12 months and confirmed by FSH level≥40 mIU/mL); or were using a highly effective method of contraception (defined as a method which resulted in a low failure rate, e.g., less than 1% per year, when used consistently and correctly, such as implants, injectables, intrauterine contraceptive devices [hormonal IUS's and non-hormonal IUDs], sexual abstinence, participant in an exclusively same-gender relationship, or a vasectomized partner). Female participants using oral contraceptives with a low failure rate (e.g., less than 1% per year) as a method of contraception were permitted to participate in this Cohort as long as they used the same regimen for the last 3 months and were not planning to make any changes in their regimen. Female participants using oral contraceptives with a low failure rate agreed to continue use and avoid egg donation for at least 90 days after the end of study participation.

Cohort 3: Participants met the criteria for Cohort 2, as well as the following: (1) Medical diagnosis of gout for at least 2 years, as confirmed by medical records. (2) A minimum of 2 acute gout flare-ups during the calendar year 2023, and no acute gout flare-ups within the last 3 months unless currently experiencing the initial symptoms of an acute gout flare-up (symptoms started≤24 hours prior to screening visit/Day 1 visit). (3) Acute gout flare-up as diagnosed by a clinical investigator (or qualified delegate). (4) If participant was receiving prophylactic gout treatment, participant was on stable prophylactic treatment for at least 6 months. Stable prophylactic treatment was defined as the same medication, dose frequency and dose strength from 6 months prior to screening and for the duration of study participation.

Efficacy assessments for Cohort 3: Measures: (1) Pain Visual Analog Scale (VAS) score; (2) Walking Disability VAS score; (3) Inflammation VAS score (visual comparison of affected joint to corresponding normal joint on opposite side of body); (4) Sleep Quality VAS score; (5) Mood Changes VAS score; (6) Treatment Satisfaction VAS score (satisfaction of study participant with study drug treatment compared to previous gout flare analgesic treatment); (7) Record of rescue medication taken by study participant during study.

### Dose Application Site

For Cohorts 1 and 2, the dose application site is one ankle per treatment period. Although the first metatarsophalangeal joint (1st MTPJ) is the most commonly affected joint during acute gouty flare, other larger joints can also be affected. The ankle joint was selected for Cohorts 1 and 2 healthy volunteer safety, tolerability, pharmacokinetic and bioavailability assessments so that investigational product exposure was increased to critically assess 125010 safety and tolerability over a large area of skin.

Using the high point of lateral malleolus as a reference point, the dosing site was the entire circumference of the ankle 3 cm above and 3 cm below this reference point.

For Cohort 1, the dose application site was switched from one ankle to the other ankle for each topical treatment period (Treatment Periods 2, 3, and 4), if both ankles met inclusion/exclusion criteria. Else, the same ankle was treated throughout all topical treatment periods.

For Cohorts 1 and 2, the dosing site was covered with Tegaderm and elastic bandage for at least 6 hours after each dose.

The dose application site in Cohort 3 was the skin surrounding the affected joint, not to exceed the average area of skin assessed in Cohorts 1 and 2. Cohort 3 participants were to apply the first dose of study medication during the Day 1 site visit under site personnel supervision. Site personnel instructed each Cohort 3 participant on the dose application site appropriate to their affected joint(s). Cohort 3 participants were instructed to leave dosing site undisturbed for approximately half an hour after dose was applied and then covered with clothing.

### Results

No clinically significant adverse events observed that were related to drug. Maximum allowable dose for this study was achieved (5.0 g per dose, 3 doses per day, 6-hour intervals).

Studies in healthy volunteers showed negligible systemic ketorolac exposure.

None of the patients used another medication to control their gout pain at any time during the study.

### Cohort 3 Results

Clinical results were assessed after a single dose or multiple doses topical administration of 5.0 g composition 125010 per dose three times daily.

Pain and walking disability improved after a single topical administration of 125010. See, FIG. 16. Based on an average change from baseline, pain (52%), walking disability (44%), and inflammation (21%) levels all demonstrated improvement.

Gout flare symptoms improved over time upon repeated topical administration of 125010 composition. See, FIG. 17. Based on an average change from baseline, pain (71%), walking disability (74%), and inflammation (62%) levels were mild after three days of treatment and nearly resolved after treatment over five days.

The clinical response rate with 125010 administration was comparable to that reported for colchicine treatment. See, FIG. 18. Three out of ten patients improved by 50% or more by 24 hours after the initial dose of study medication (comparable to what is reported for low dose colchicine (37.8%) and high dose colchicine (32.7%)). See, e.g., Terkeltaub, R. A. et al. Arthritis and Rheumatism, 2010, vol. 62 (4), pages 1060-1068.

Eight out of 10 participants expressed an improvement in 125010 treatment satisfaction over their prior gout treatment. Five out of 10 participants improved their treatment satisfaction over 80%. See, FIG. 19 and Table 4.

TABLE 4
Treatment Satisfaction Scores for Prior Gout Treatment Compared to
Composition 125010

| | Prior Treatment | 125010 Treatment | |
|---|---|---|---|
| | Satisfaction | Satisfaction | Difference |
| mean | 47.7 | 64.1 | 16.4 |
| Std. Dev. | 26.5 | 27.2 | 40.9 |
| 95% confidence | (28.8, 66.6) | (44.6, 83.6) | (−12.8, 45.6) |
| interval | | | |

Paired t-test for mean difference: p = 0.24, 2-sided;
Wilcoxon Signed-Rank test for median difference: p = 0.27, 2-sided.

With respect to pain score, 125010 (“NOV-1776”) performed well compared to gout pain standards of care low dose colchicine and naproxen. See, FIG. 20.

Participants reported reduction in gout flare duration. See, FIG. 21. Days with moderate to severe pain were reduced for 8 out of 10 participants, with an average of 46% duration reduction. Days with moderate to severe inflammation were reduced for 7 out of 10 participants, with an average of 56% duration reduction. At the time of study exit, Participants 303 and 310 had moderate swelling, with VAS scores of 34 and 38, respectively.

A potential trend was observed in flare duration estimates for repeat dosing of composition 125010 compared with participant experiences in managing gout flares before study participation:

| | |
|---|---|
| 4 days | Median self-reported gout flare duration for flares |
| | occurring prior to study participation |
| 2.75 days | Median days required for pain, inflammation and |
| | walking disability VAS scores to return to ≤ 30. |
| 2 days | Median paired difference |
| p-value | 0.07 (based on Wilcoxon Signed-Rank test, 2-sided) |

Ketorolac plasma concentrations were significantly lower than that observed for those treated with ketorolac intravenous dosing in a comparative single day dosing regimen. See, FIG. 22A. In repeat dosing over five days, topical administration of 125010 continued to afford low plasma levels of ketorolac with no appreciable accumulation observed. See, FIG. 22B.

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A composition comprising:
(a) from about 1% to about 50% (w/w) ketorolac or pharmaceutically acceptable salt thereof;
(b) from about 5% to about 50% (w/w) choline chloride; and
(c) from about 20% to about 70% (w/w) propylene glycol; and
(d) from about 10% to about 30% (w/w) dimethyl sulfoxide;
wherein the choline chloride and the propylene glycol form a mixture having a lower melting point than pure propylene glycol.

2. The composition of claim 1, comprising from about 1% to about 40% (w/w) ketorolac or pharmaceutically acceptable salt thereof.

3. The composition of claim 1, wherein the ketorolac or pharmaceutically acceptable salt thereof comprises ketorolac tromethamine.

4. The composition of claim 1, comprising from about 5% to about 40% (w/w) choline chloride.

5. The composition of claim 1, comprising from about 20% to about 60% (w/w) propylene glycol.

6. The composition of claim 1, wherein the choline chloride and the propylene glycol is present in an amount of from about 5:2 to about 1:10 by weight.

7. The composition of claim 1, comprising from about 10% to about 30% dimethyl sulfoxide.

8. The composition of claim 1, comprising:
(a) from about 10% to about 15% (w/w) ketorolac tromethamine;
(b) from about 15% to about 25% (w/w) choline chloride;
(c) from about 35% to about 45% (w/w) propylene glycol; and
(d) from about 15% to about 25% (w/w) dimethyl sulfoxide.

9. The composition of claim 1, comprising:
(a) about 12.5% (w/w) ketorolac tromethamine;
(b) about 20% (w/w) choline chloride;
(c) about 40% (w/w) propylene glycol; and
(d) about 20% (w/w) dimethyl sulfoxide.

10. The composition of claim 1, further comprising an emulsifier.

11. The composition of claim 1, wherein the emulsifier comprises a surfactant.

12. The composition of claim 11, wherein the emulsifier comprises polyethylene glycol monododecyl ether.

13. A method of treating inflammation or pain in a human subject in need thereof, comprising topically administering to the subject a composition comprising:
(a) from about 1% to about 50% (w/w) ketorolac or pharmaceutically acceptable salt thereof;
(b) from about 5% to about 50% (w/w) choline chloride; and
(c) from about 20% to about 70% (w/w) propylene glycol; and
(d) from about 10% to about 30% (w/w) dimethyl sulfoxide;
wherein the choline chloride and the propylene glycol form a mixture having a lower melting point than pure propylene glycol.

14. The method of claim 13, comprising treating inflammation.

15. The method of claim 13, wherein the inflammation comprises an inflammation of the joint.

16. The method of claim 13, wherein the inflammation comprises gout.

17. The method of claim 13, comprising treating pain.

18. The method of claim 13, wherein the pain comprises post-operative pain.

19. The method of claim 13, comprising topically administering once, twice, or three times daily.

20. A method of treating inflammation or pain associated with acute gout in a human subject in need thereof, comprising topically administering three times daily to a joint in the subject a therapeutically effective amount of a composition comprising:
(a) from about 1% to about 50% (w/w) ketorolac or pharmaceutically acceptable salt thereof;
(b) from about 5% to about 50% (w/w) choline chloride; and
(c) from about 20% to about 70% (w/w) propylene glycol; and
(d) from about 10% to about 30% (w/w) dimethyl sulfoxide;
wherein the choline chloride and the propylene glycol form a mixture having a lower melting point than pure propylene glycol.

21. The method of claim 20, wherein the composition comprises:
(a) about 12.5% (w/w) ketorolac tromethamine;
(b) about 19.5% (w/w) choline chloride;
(c) about 39.4% (w/w) propylene glycol;
(d) about 19.6% (w/w) dimethyl sulfoxide;
(e) about 7.0% (w/w) glyceryl stearate;
(f) about 0.2% (w/w) sorbitan monooleate; and
(g) about 1.8% (w/w) polysorbate 80.

22. The method of claim 20, wherein the composition is administered in about 5.0 g per dose.

23. The method of claim 22, wherein the composition is administered for at least about 1 day, at least about 2 days, at least about 3 days, at least about 4 days, or for at least about 5 days.

24. The method of claim 23, wherein less than about 1 μg/mL, less than about 0.1 μg/mL, or less than about 10 ng/mL ketorolac is measured in plasma from the subject after topical administration.
